(19) United States
(12) Patent Application Publication
(10) Pub. No.: US 2024/0350649 A1
(43) Pub. Date: Oct. 24, 2024

(54) TREATMENT AND DIAGNOSIS OF IMMUNE DISORDERS RELATING TO ABERRANT IMMUNE CELLS

(71) Applicant: Octagon Therapeutics, Inc., Boston, MA (US)

(72) Inventors: Luis Rolando Castillo-Menendez, Chelsea, MA (US); Nathaniel Sherden, Boston, MA (US); Brent Cezairliyan, Boston, MA (US); Shen Yu, Winchester, MA (US)

(21) Appl. No.: 18/294,879

(22) PCT Filed: Aug. 5, 2021
(86) PCT No.: PCT/US21/44638

§ 371 Date: Feb. 2, 2024

(51) Int. Cl. A61K 47/68; A61K 31/7105; C07K 16/28; C12N 9/64

(52) U.S. Cl. CPC A61K 47/68; A61K 31/7105; C07K 16/28; C12N 9/6491; C12Y 304/24; C12N 2310/11; C12N 2310/141

(57) ABSTRACT

Disclosed are compositions and methods of treating and diagnosing immune-related disorders characterized by the presence of aberrant immune cells that over-express certain proteins or that express a protein not expressed on normal immune cells.

### FIELD OF THE INVENTION

The present invention is in the field of medicine. More particularly, the invention relates to the diagnosis and treatment of immune diseases and disorders relating to aberrant immune cells.

### BACKGROUND OF THE INVENTION

The immune system plays essential roles in maintaining health as it defends the host from the infection of pathogens. However, when it malfunctions or when led to the wrong target, the immune system is responsible for causing various immune-related disorders such as autoimmune diseases and inflammatory diseases.

Various protein markers produced in response to autoimmune disorders and inflammation are useful as disease markers. For example, various serum proteins called acute phase reactants are produced in response to inflammation. Pro-inflammatory cytokines such as IL-1, IL-6, and TNF-alpha induce synthesis of some acute phase reactants that include CRP, fibrinogen and haptoglobin. Other proteins, like serum albumin, are sensitive to chronic stress (inflammation) which causes a lower synthesis rate with resultant decreased serum concentrations.

The first generation therapies for immune-related disorders demonstrating clinical utility were antiproliferative agents such as cancer chemotherapy agents and immunosuppressants. These compounds non-selectively affect rapidly proliferating cells, usually by interfering with cellular metabolism, and thereby exhibit a range of toxicities including bone marrow suppression, GI-toxicity (affecting intestine epithelial cells or crypt cells), and hair loss (affecting hair follicles). Rapidly-proliferating lymphocytes are important for normal immune function and are critical in controlling infection and in cancer surveillance. Non-selective suppression of lymphocytes exposes patients to elevated risk of opportunistic infections as well as neoplasia.

Biologic therapies based on highly selective monoclonal antibodies that modulate specific inflammatory or effector pathways have been used to treat autoimmune disorders (e.g., TNF inhibitors, IL-6 inhibitor, IL-12 inhibitor, BLyS inhibitor CTLA4-IgGs and anti-CD20 antibodies).

Unfortunately, many biologic therapies for autoimmune diseases have significant drawbacks. For example, the anti-CD20 antibody, Rituximab (Rituxan), widely used to treat B-cell malignancies as well as autoimmune diseases, triggers cell death via antibody-dependent cellular cytotoxicity (ADCC) when it binds to CD20 on a B-cell surface. However, Rituximab is known to cause headache and back pain, in addition to possessing a slow administration infusion rate (50 mg/hr), which in practice means that it can take up to eight hours to administer a standard dose. In addition, its administration increases the risk of infections and malignancies as it depletes B cells; normal B-cell functions are essentially absent for patients treated with Rituximab. Treatments like Intravenous Immune Globulin (IVIG) can partially restore B-cell functions, but the method has severe toxicity liabilities.

In autoimmune disorders like rheumatoid arthritis, anti-TNF biologic drugs such as Adalimumab (Humira, also used in other autoimmune disorders such as inflammatory bowel diseases (IBD) and psoriasis) are often used as front-line treatment. Unfortunately, anti-TNF biologics generally suffer from low response rate, elevated risk for tumors and infections and pain at injection site. For these reasons, it is often necessary to combine biologic drugs with other small molecule chemotherapeutic agents or disease-modifying antirheumatic drugs (DMARDs) to achieve satisfactory therapeutic effects. Examples include combination of Rituximab with four chemotherapeutic agents (known as the R-CHOP regimen, Rituximab with cyclophosphamide, hydroxydaunorubicin, oncovin, prednisone/prednisolone) in the treatment of non-Hodgkin lymphomas, and combination of adalimumab with methotrexate in the treatment of RA. However, when combined with these non-selective small molecule drugs, the advantage of cell-selectivity of the antibodies is lost.

Mycophenolate mofetil (MMF, Cellcept) was developed as an immunosuppressant. It is widely used to prevent allograft rejection and is used off-label in a variety of autoimmune diseases. MMF is a prodrug of mycophenolic acid (MPA) that increases the bioavailability of MPA which inhibits inosine monophosphate dehydrogenase (IMPDH). However, MMF suffers from poor PK properties and significant GI toxicity. Several other IMPDH inhibitors have been developed as anti-proliferative agents for select autoimmunity and cancer indications (the Cortellis database) but were abandoned late in clinical development due to off-target safety concerns.

Despite recent advances in the understanding of molecular mechanisms of diseases caused by disorders of the immune system, there remains significant unmet need for treatments and diagnostic markers for immune-related disorders.

### SUMMARY OF THE INVENTION

It has been discovered that the plasma or serum of autoimmune patients are able to alter otherwise normal immune cells to aberrant immune cells, and that these aberrant immune cells express certain enzymes/receptors or exhibit higher levels of enzymatic activities/receptor-mediated signaling compared to normal immune cells. It has also been discovered that in certain immune-related disorders and diseases, there is high correlation between immune disease activity and an increase in certain immune cell-related enzymatic activity.

These discoveries have been exploited to develop the present invention, which, in part, is directed to compositions for, and methods of, treating immune disorders by selectively targeting dysregulated, aberrant immune cells using at least one aberrant cell-expressed enzyme- or receptor-specific ligand (“Bait”) attached to a therapeutic Payload.

In one aspect, the disclosure provides an aberrant immune cell-selective compound, or a pharmaceutically acceptable salt thereof, comprising: a Bait comprising a ligand moiety that binds an enzyme or receptor that binds an enzyme or receptor overexpressed by the aberrant immune cell relative to a normal immune cell, or is not expressed or is expressed at lower levels by a normal immune cell, or having more activity than the enzyme or receptor has in a normal immune cell, the aberrant immune cell being associated with an immune-related disorder; and a Payload comprising a therapeutic moiety that reduces an activity of, inhibits the proliferation of, or kills, the aberrant immune cell.

In a particular embodiment, the compound comprises a Bait that binds to a matrix metalloprotease on or in the aberrant immune cell.

In some embodiments, the compound comprises a Bait comprising a Substrate of the enzyme or that binds to the receptor. In certain embodiments, the Bait comprises multiple Substrates in tandem. In specific embodiments, the Substrate comprises a peptide bond.

In some embodiments, the compound has the structure Bait----Payload, wherein the Bait comprises a Cap and from about one to about five Subunits. In specific embodiments, the Cap is selected to the group consisting of

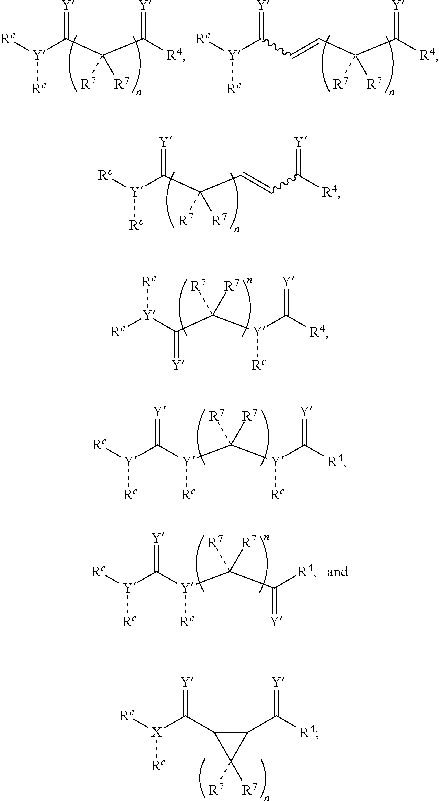

wherein R⁴ is independently, at each occurrence, selected from the group consisting of a bond to any R⁸ or to any R⁶ position in a Subunit.

In certain embodiments, the Subunit is selected to the group consisting of

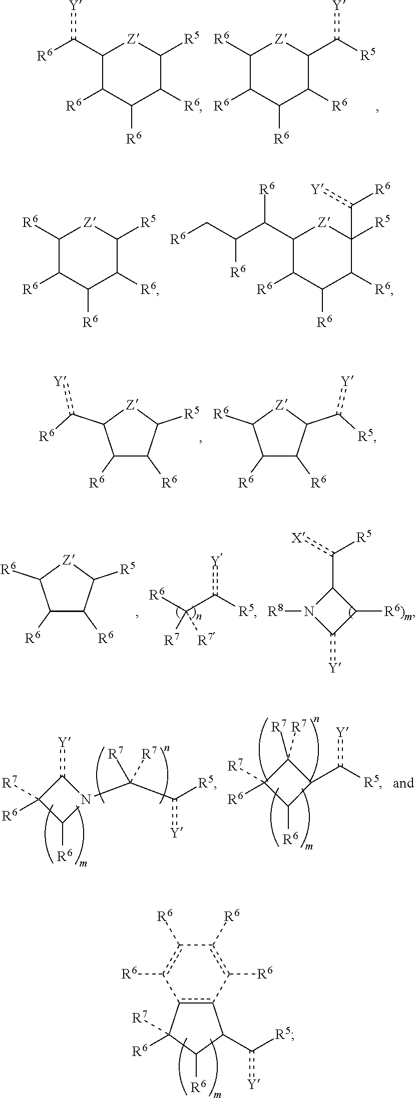

wherein:
--- is an optional single bond;
=== is an optional single or double bond;
Z′ is independently, at each occurrence, selected from the group consisting of CH₂, NH, O, and S;
R⁵ is independently, at each occurrence, selected from the group consisting of a bond to an R⁶ in another Subunit or to a Subunit R⁸ in another Subunit, and a bond to the Payload,
X′ and Y′ are independently, at each occurrence, selected from the group consisting of O, NH, and S;
R⁶ is independently, at each occurrence, selected from the group consisting of H, —R^{D}, —R^{A}—R⁵ when R⁵ is in another Subunit, —R^{A}—R⁴ when R⁴ is in a Cap, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo(s);
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁₀ aryl, a 5-, 6-, 7-, 8-, 9-, or 10-membered heteroaryl, C₁₋₄ alkyl, —(CH₂)₁₋₃-C₆₋₁₀ aryl, and —(CH₂)₁₋₃-5-, 6-, 7-, 8-, 9-, or 10-membered heteroaryl, wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{c}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵ in another Subunit, or a bond to R⁴ inn a Cap;
m is 0, 1, 2, 3, or 4; and
n is 0, 1, 2, or 3.

In some embodiments, the compound comprises a Bait which comprises a prodrug.

In certain embodiments of the compound, the Bait comprises a substrate of an MMP, the substrate comprising N-succinyl-alanine-alanine-phenylalanine (SAAF), N-glutaryl-alanine-alanine-phenylalanine (GAAF)-, N-succinyl-alanine-alanine-leucine (SAAL)-, N-glutaryl-alanine-alanine-leucine (GAAL), N-malonyl-alanine-alanine-phenylalanine (MAAF), r N-malonyl-alanine-alanine-leucine (MAAL), alanine-alanine-phenylalanine (AAP), alanine (A), methionine (M), 7-methoxy, glycine-R, or L-leucine.

In other embodiments, the compound comprises a Bait that comprises an immunoglobulin or binding fragment thereof. In certain embodiments, the immunoglobulin is an antibody or specific binding fragment thereof, a camelid, a single domain immunoglobulin, a chimeric antigen receptor (CAR), or a CAR T cell. In particular embodiments, the antibody is a human antibody, a recombinant antibody, a humanized antibody, a bispecific antibody, or a monoclonal antibody.

In some embodiments, the compound comprises a Bait that comprises more than one ligand, e.g., a ligand conjugated to a second and/or more ligands. In specific embodiments, the second and/or additional ligand(s) is/are specific for a protein on or in the aberrant immune cells that is different than the enzyme or receptor to which the first ligand binds. In some embodiments, the compound comprises a Bait that is a composite of one or more different enzyme or receptor ligands. In such embodiments, each ligand in the composite Bait is specific for a protein on a target aberrant immune cell, the protein being different than the enzyme or receptor for other ligands in the composite. Thus, in some instances, a Bait made of a composite of ligands may use a sequential series of enzyme/receptor activations to release the Payload.

In some embodiments, the compound comprises a Payload which comprises a therapeutic moiety comprising a small molecule. In certain embodiments, the small molecule is a metabolic inhibitor, a small molecule cytotoxic agent, or a small molecule inhibitor of an immune cell receptor. In particular embodiments, the therapeutic moiety comprises a prodrug.

In other embodiments, the compound comprises a Payload comprising a therapeutic moiety comprising a polynucleotide that inhibits or reduces the expression of an immune cell metabolic enzyme or receptor. In specific embodiments, the polynucleotide is an antisense polynucleotide, an miRNA, or an siRNA.

In yet other embodiments, the compound comprises a Payload comprising a therapeutic moiety that comprises an immunoglobulin, or binding fragment thereof. In certain embodiments, the immunoglobulin comprises an antibody, a camelid, a single domain immunoglobulin, or a CAR. In particular embodiments, the antibody comprises a human antibody, a recombinant antibody, a humanized antibody, a monoclonal antibody, a bispecific antibody, a monoclonal antibody, an antibody-drug conjugate, or a binding fragment thereof. In certain embodiments, the antibody is an anti-TNF antibody, an anti-CD19 antibody, anti-CD22 antibody, an anti-CD80 antibody, an anti-CD86 antibody, an anti-IL-6 receptor antibody, an anti-Interleukin-1 type I receptor (IL-1RI) antibody, an anti-IL-12/23 antibody, an anti-CD20 antibody, or an anti-IL-17 antibody.

In some embodiments, the immunoglobulin or binding fragment thereof, is conjugated to a second therapeutic moiety. In a particular embodiment, the immunoglobulin comprises an antibody-drug conjugate. In certain embodiments, the second therapeutic moiety comprises a small molecule cytotoxic agent, a small molecule inhibitor of a metabolic enzyme or receptor, or a small molecule inhibitor of an immune cell receptor. In other embodiments, the immunoglobulin is conjugated to a second immunoglobulin or binding fragment thereof. In specific embodiments, the second immunoglobulin is a human antibody, a recombinant antibody, a humanized antibody, a bispecific antibody, a monoclonal antibody, a camelid, or a single domain immunoglobulin. In particular embodiments, the second immunoglobulin is an anti-TNF antibody, an anti-CD19 antibody, an anti-CD22 antibody, an anti-CD80 antibody, an anti-CD86 antibody, an anti-IL-6 receptor antibody, an anti-Interleukin-1 type I receptor (IL-1RI) antibody, an anti-IL-12/23 antibody, an anti-CD20 antibody, or an anti-IL-17 antibody.

In another aspect, the disclosure provides a formulation comprising the compound of any of the above-described therapeutic compounds, and a pharmaceutically acceptable carrier.

In still another aspect, the disclosure provides a method of treating, or decreasing a risk of protracting, an immune-related disorder in a subject, comprising administering to the subject an amount of any of the compounds of formulation described above, effective to reduce a symptom of, or to reduce the risk of protracting, the immune-related disorder. In some embodiments, the immune-related disorder is an inflammatory disorder. In certain embodiments, the disorder is systemic lupus erythematosus (SLE), rheumatoid arthritis (RA), multiple sclerosis (MS), pemphigus vulgaris, bullous pemphigoid, scleroderma, myasthenia gravis, psoriasis, autoimmune myositis, or infection by SARS-CoV2.

The disclosure also provides use of the aberrant immune cell-selective compound, or a pharmaceutically acceptable salt thereof, as described above, or the pharmaceutical formulation comprising the same as described above, to reduce a symptom of an immune-related disorder in a subject, or to reduce the risk of a subject contracting an immune-related disorder.

### DESCRIPTION OF THE DRAWING

The foregoing and other objects of the present disclosure, the various features thereof, as well as the disclosure itself may be more fully understood from the following description, when read together with the accompanying drawings in which:

FIG. 1 is a schematic representation of a therapeutic compound according to the disclosure;

FIG. 2A is a diagrammatic representation of a nonlimiting preparation of a Bait or Substrate, according to the disclosure, wherein when a Substrate is e built up from commercially available units, this is done in a sequential manner of coupling and deprotection, and where a Substrate ready to be attached to a Payload directly, this is done via a single R5 position that is the point of attachment to Payload, which is left un-protected (labeled as “Y);

FIG. 2B is a diagrammatic representation of the final assembly of a representative compound according to the disclosure, where all boxed entities represent chemical mater—either a whole molecule or where joined by a line represent a portion of a molecule connected by one or more chemical bonds to the other portion(s) as indicated by the connecting line; arrows indicate actions such as synthetic step(s), solid lines for both boxes and arrows represent required steps and molecules or portions thereof, and dashed lines for boxes and arrows represent steps and molecules or portions thereof that are conditional;

FIG. 3A is a representation of a scatter plot showing the expression level of CD10 (as one non-limiting example of the MMPs as targeting groups) on primary B cells when the cells were stimulated with plasma from a healthy donor, where the four quadrants separate the cell population into CD19−CD10− (bottom left), CD19+CD10− (upper left), CD19−CD10+ (bottom right), and CD19+CD10+ (upper right) populations using standard cutoff values;

FIG. 3B is a representation of a scatter plot showing the expression level of CD10, on primary B cells when the cells were stimulated with plasma from a pemphigus vulgaris (PV) donor, where the four quadrants separate the cell population into CD19−CD10− (bottom left), CD19+CD10− (upper left), CD19−CD10+ (bottom right), and CD19+CD10+(upper right) populations using standard cutoff values;

FIG. 3C is a representation of a scatter plot showing the expression level of CD10 on primary B cells when the cells were stimulated with plasma from a SLE donor, where the four quadrants separate the cell population into CD19−CD10− (bottom left), CD19+CD10− (upper left), CD19−CD10+ (bottom right), and CD19+CD10+ (upper right) populations using standard cutoff values;

FIG. 4A is a schematic representation of the chemical structure of OTG177 as an example of a compound according to the disclosure that contains a Bait as the targeting group (SAAF, which can be hydrolyzed by CD10), a linker moiety, and an IMPDH inhibitor Payload (ribavirin-monophosphate, RMP);

FIG. 4B is a graphic representation of the antiproliferative activity of OTG177 on CD19+ primary B cells stimulated with a healthy donor's plasma, as measured by a standard BrdU proliferation kit;

FIG. 4C is a graphic representation of the antiproliferative activity of OTG177 on CD19+ primary B cells stimulated with a flaring pemphigus vulgaris (PV) donor's plasma, measured by a standard BrdU proliferation kit;

FIG. 4D is a graphic representation of the antiproliferative activity of OTG177 on CD3+ primary T cells stimulated with a healthy donor's plasma, measured by a standard BrdU proliferation kit;

FIG. 4E is a graphic representation of the antiproliferative activity of OTG177 on CD3+ primary T cells stimulated with a flaring pemphigus vulgaris donor's plasma, measured by a standard BrdU proliferation kit;

FIG. 5A is a representation of a scatter plot showing the gating strategy to examine peripheral blood mononuclear cell (PBMC) composition (granulocyte, monocyte, lymphocyte) by flow cytometry, where PBMCs were gated by side scattering (SSC) vs. CD45 (blue fluorescent channel), R2 represents lymphocytes, R5 represents monocytes, and R6 represents granulocytes;

FIG. 5B is a graphic representation showing that OTG177 does not have obvious effects on the % population of PBMC cells (granulocytes, monocytes, and lymphocytes at up to 50 μM concentration stimulated by healthy donor plasma, PV donor plasma, or SLE donor plasma;

FIG. 5C is a representation of a scatter plot showing the results of the gating strategy used to examine primary B and T lymphocytes from a PBMC population, where PBMCs were gated by forward scattering (FSC) vs. side scattering (SSC), where R3 represents B and T lymphocytes;

FIG. 5D is a representation of a scatter plot showing the results of the gating strategy to examine primary B and T lymphocytes from group R3 in FIG. 5C, where the R3 population was further gated by CD19 in the red channel vs. CD3 in the green channel, and R4 and R8 represent B and T cells, respectively;

FIG. 5E is a graphic representation showing that OTG177-exhibited, dose-dependent (1 μm, 10 μm, 50 μm, DMSO control), suppressive effect on primary CD19+ B lymphocytes in a mixed PBMC population, when the PBMCs are stimulated with plasma from either a flaring pemphigus vulgaris (PV) donor, or a flaring systemic lupus erythematosus (SLE) donor, but not a healthy donor; and

FIG. 5F is a graphic representation showing that OTG177 at 1 μm, 10 μm, 50 μm, or 0 (DMSO control), exhibited no obvious effect on primary CD3+T lymphocytes in a mixed PBMC population, when the PBMCs are stimulated with plasma from either a flaring pemphigus vulgaris (PV) donor, a flaring systemic lupus erythematosus (SLE) donor, or a healthy donor.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.10% from the specified value, as such variations are appropriate to perform the disclosed methods.

The term “treat,” “treated,” “treating,” or “treatment” includes the diminishment or alleviation of at least one symptom associated or caused by the immune-related disorder or disease being treated. The term “therapeutic” as used herein means a treatment and/or prophylaxis. A therapeutic effect is obtained by suppression, remission, or eradication of a disease state.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a decrease or complete suppression of at least one symptom. The skilled artisan would understand that the amount of the therapeutic composition or formulation administered varies and can be readily determined based on a number of factors such as the disease or condition being treated, the age and health and physical condition of the mammal being treated, the severity of the disease, the particular compound being administered, and the like.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of a ligand such as an antibody, a protein, or a peptide, with a second chemical species, to mean that the interaction is dependent upon the presence of a particular structure (e.g., an antigenic determinant or epitope) on the chemical species; for example, an antibody recognizes and binds to a specific protein structure rather than to proteins generally.

The terms “patient” and “subject” as used herein, refer to the recipient of a method as described herein, i.e., a recipient that can mount a cellular immune response, and is a mammal. In certain embodiments, the subject is a human. In certain embodiments, the subject is a domesticated animal, e.g., a horse, a cow, a pig, a sheep, a dog, a cat, etc. The terms “patient” and “subject” may be used interchangeably. As used herein, the subject is an animal which is or may be suffering from an immune-related disorder.

The term “donor” refers to a mammalian subject, e.g., a human, from which a bodily sample, e.g., plasma or blood, is taken and provided to a different subject.

The terms “disorder” and “disease” are used interchangeable when referring to illnesses and conditions relating to the immune system and response.

The term “immune cell” refers to a cell that is part of the immune system and that under normal conditions, helps the body fight infections and other diseases. These cells include, but are not limited to, lymphocytes (T-cells, B-cells and NK cells), neutrophils, and monocytes/macrophages.

The term “aberrant immune cell” encompasses immunes cells that are associated with immune-related disorders and that over-express a receptor or metabolic enzyme, or that exhibit a higher degree of a specific enzymatic activity, or that express a receptor or enzyme that normal immune cells do not express, or that possess an enzymatic activity, that normal immune cells do not have.

A “normal immune cell” is an immune cell that performs its normal immunological functions and is not contributing to an immune disorder.

As used herein, the term “chimeric T cell receptor” or “CAR” refers to a complex of membrane proteins that participate in the activation of T cells in response to the presentation of antigen. The T cell receptor recognizes antigens bound to major histocompatibility complex molecules. It is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. The TCR may be modified on any cell comprising a TCR, including, for example, a helper T cell, a cytotoxic T cell, a memory T cell, regulatory T cell, natural killer T cell, and gamma delta T cell. The CAR on the T cell is referred to as a “CAR T”.

Amounts throughout this disclosure, various aspects of the disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, the term “pharmaceutically acceptable” refers to a material, such as a carrier or diluent, which does not abrogate the biological activity or properties of the cell-targeting enzyme/receptor-ligand or substrate-binder, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition” refers to a molecule which has a therapeutic activity relating to immune disorders and/or which can be used to detect the cell-targeting enzyme/receptor and/or in in the preparation of a therapeutic formulation.

The terms “pharmaceutical composition”, or “formulation” refers to a molecule or combination thereof including a carrier for diagnostic or therapeutic use.

An “oral dosage form” includes a unit dosage form prescribed or intended for oral administration.

The term “Bait” refers to a moiety or component of the therapeutic compound according to the disclosure which encompasses one or more binding moiety or ligand which specifically binds to a targeted protein or proteins on, in, or excreted by, an aberrant immune cell associated with an immune-related disorder, and which may be labeled to identify such aberrant immune cells. This component optimizes the compound to achieve aberrant immune cell-selective targeting and accumulation by serving, for example, as a substrate for the targeted enzyme(s) or which binds the targeted receptor(s) present on or in the targeted aberrant immune cell. A Bait may comprise one or more subunits which may be the same or have a different structure. Linkages to the subunits are in some instances to another subunit of the same structure, and in other instances, to another subunit (s) having a different structure.

The term “proBait” refers to a moiety of a molecule that can be processed by the host's metabolism to a moiety that matches the definition above for the term Bait.

The term “Payload” encompasses a therapeutic component or moiety of the therapeutic compound according to the disclosure that is attached to the Bait, which can treat, inhibit, or kill the aberrant immune cell associated with an immune-related disorder, and which may be labeled to identify such aberrant immune cells.

The term “Cap” refers to an optional molecule terminating the Bait and attached to a Subunit. The Cap comprises a small organic molecule such as, but not limited to, a carboxylic acid, urea, carbamate, or sulfonate, including, but not limited to, those existing in nature.

The term “Subunit” refers to a small organic molecule making up at least part of the Bait and which may be an amino acid, sugar, and/or lipid, including, but not limited to, those existing in nature.

The term “antibody” is used to mean an immunoglobulin molecule that recognizes and specifically binds to a target or antigen, such as a protein, polypeptide, peptide, carbohydrate, polynucleotide, lipid, or combinations of the foregoing etc., through at least one antigen recognition site within the variable region of the immunoglobulin molecule. As used, herein, the term encompasses intact polyclonal antibodies, intact monoclonal antibodies, antibody fragments, single chain Fv (scFv) mutants, multispecific antibodies such as bispecific antibodies generated from at least two intact antibodies, monovalent or monospecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively, and can also be a camelid.

As used herein, the term “antibody fragment” refers to a portion of an intact antibody and refers to the antigenic determining and binding variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

The term “expression” or “expresses” are used herein to refer to transcription and translation occurring within a host cell, e.g., the expression of CD10 by a B cell or the expression of a CAR by a T cell. The level of expression of a product gene in a host cell can be determined on the basis of either the amount of corresponding mRNA that is present in the cell or the amount of the protein encoded by the product gene that is produced by the cell.

The term “hybridoma” as used herein refers to a cell created by fusion of an immortalized cell derived from an immunologic source and an antibody-producing cell. The resulting hybridoma is an immortalized cell that produces antibodies. The individual cells used to create the hybridoma can be from any mammalian source, including, but not limited to, rat, pig, rabbit, sheep, pig, goat, and human. The term also encompasses trioma cell lines which result when progeny of heterohybrid myeloma fusions, which are the product of a fusion between human cells and a murine myeloma cell line, are subsequently fused with a plasma cell. Furthermore, the term is meant to include any immortalized hybrid cell line that produces antibodies such as, for example, quadromas (See, e.g., Milstein et al., Nature, 537:3053 (1983)).

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. In an embodiment, C₁-C₆ alkyl groups are provided herein. Nonlimiting examples include methyl, ethyl, propyl, isopropyl, butyl, isobutyl, tert butyl, pentyl, neopentyl, and hexyl. Other nonlimiting examples of C₁-C₆ alkyl include ethyl, methyl, isopropyl, isobutyl, n-pentyl, and n-hexyl.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. For example, the alkenyl moiety contains 2 to 6, 2 to 4, or 2 to 3 carbon atoms. Exemplary alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “allyl” employed alone or in combination with other terms, refers to an sp³ carbon atom adjacent to an alkenyl group as defined above.

As used herein, the term “halo” or “halogen” alone or as part of another substituent means, unless otherwise stated, a fluorine, chlorine, bromine, or iodine atom.

As used herein, the term “aromatic” refers to a carbocycle or heterocycle with one or more polyunsaturated rings and having aromatic character, i.e., having (4n+2) delocalized π elections where n is an integer).

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. Aryl groups may have 6 carbon atoms, six to ten carbon atoms, or six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclo-penta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]-triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl and heteroaryl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, and the term “thienyl” means 2- or 3-thioenyl.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The present disclosure provides methods of treating, diagnosing, and preventing immune-related disorders associated with the presence of aberrant immune cells, and compositions and formulations for treating, diagnosing, and preventing such disorders. Aberrant immune cells associated with certain immune-related disorders express certain proteins, such as certain receptors and metabolic enzymes, that a normal immune cell does not express, or which over-express such proteins relative to the expression in normal immune cells, or which exhibit a higher degree of a specific enzymatic activity relative to normal immune cells. The compositions according to the disclosure target these aberrant immune cells by binding to these proteins and treating or identifying and treating the aberrant immune cells with a therapeutic moiety.

### Aberrant Immune Cells

The present disclosure demonstrates that the plasma or serum of autoimmune patients are able to alter otherwise normal immune cells to aberrant immune cells, and that these aberrant immune cells express certain enzymes/receptors or exhibit higher levels of enzymatic activities/receptor-mediated signaling compared to normal immune cells.

Immune cells and their immune phenotypes that are targeted for diagnosis and/or treatment according to the disclosure include, but are not limited to, those in Table 1 below.

TABLE 1

| | Immune Phenotype |
|---|---|
| Immune Cells Populations | |
| B cell | CD19⁺ |
| “Helper” T cells | CD4⁺CD8⁻ |
| “Effector” T cells | CD8⁺CD4⁻ |
| Natural Killer (NK) Cells | CD16⁺CD56⁺ |
| Mast Cells | CD23⁺CD117⁺ |
| Neutrophils | CD10⁺ CD13⁺ CD15⁺ CD33⁺ CD141⁺ CD178⁺ |
| Basophils | CD9⁺ CD23⁺ CD24⁺ CD32⁺ CD43⁺ CD69⁺ |
| Eosinophils | CD9⁺ CD23⁺ CD24⁺ CD32⁺ CD43⁺ CD49d⁺ |
| Monocytes | CD2⁺ CD11b⁺ CD14⁺ CD31⁺ CD62L⁺ CD115⁺ CD192⁺ |
| Macrophages | CD14⁺ CD16⁺ CD64⁺ CD68⁺ CD71⁺ CCR5⁺ |
| B Cell Subset | |
| Transitional | CD20⁺ CD27⁻ CD38^{hi} IgM⁺ CD24^{hi} BR3⁺ |
| Follicular | IgM^{lo} CD23⁺ CD93⁻ CD19⁺ CD20⁺ CD21⁺ CD22⁺ |
| Marginal zone | IgM^{hi} IgD^{lo} CD1c⁺ CD24⁺ CD19⁺ CD20⁺ CD21⁺ |
| Germinal center | CD20⁺ CD38⁺ BR3⁺ IgD⁻ |
| Plasma cells | CD20⁻ CD38^{hi} CD27^{hi} CD138⁺ TACI⁺ and/or |
| | BCMA⁺ CD126⁺ CD319⁺ CD78⁺ |
| Memory B Cell | CD20⁺ CD38⁻ CD27⁺ CD80⁺ CD84⁺ CD86⁺ |
| T Cell Subset | |
| CD8 Memory T cell | CD3⁺ CD8⁺ CD45RO⁺ |
| CD8 cytotoxic T cell | CD3⁺ CD8⁺ Granzyme A/B⁺ |
| Treg | CD25⁺ CD39⁺ CD73⁺ CD103⁺ |
| CD4 memory T Cell | CD3⁺ CD4⁺ CD25⁺ CD45RO⁺ CD62L⁺ |
| Th1 | CD178⁺ CD183⁺ CD195⁺ CD212⁺ CD218α⁺ |
| Th2 | CD183⁺ CD193⁺ CD194⁺ CD198⁺ CD294⁺ |
| Th9 | CD196⁺ IL-9⁺ |
| Th17 | CD45RO⁺ CD126⁺ CD161⁺ CD194⁺ CD196⁺ IL-13α1⁺ |
| | IL-21R⁺ |
| Th22 | CCR10⁺ CD140a⁺ CD140b⁺ CD194⁺ CD196⁺ |
| Tfh | CD185⁺ CD279⁺ |
| Macrophage Subset | |
| M1 | CD68⁺ CD86⁺ CD282⁺ CD284⁺ |
| M2 | CD163⁺ CD200R⁺ CD206⁺ |
| TAM | CD81⁺ CD106⁺ Dectin-1⁺ VEGF⁺ |
| CD169⁺ macrophage | CD106⁺ CD169⁺ CD206⁺ |
| TCR⁺ macrophage | CD3⁺ ZAP70⁺ TCRα/β⁺ |

### Targeted Aberrant Cell Proteins

Proteins targeted and bound by the compositions according to the disclosure include receptors and enzymes. Aberrant immune cells can express these proteins that normal immune cells do not usually express, or can over-express such proteins on their cell surface or within their cytoplasm. Aberrant immune cells can also potentially exhibit increased protein (e.g., enzymatic or receptor-related) activity toward a substrate if the substrate and targeted protein are co-localized to a greater extent than in healthy immune cells. This could be due to increased production of endocytic vesicles in aberrant immune cells, resulting in trapping of substrate in intracellular vesicles containing both substrate and the targeted protein at high concentrations.

Useful targeted aberrant immune cell enzymes include, but are not limited to, matrix metalloproteases (MMPs). MMPs, also known as matrix metalloproteinases or matrixins, are calcium-dependent, zinc-containing endopeptidases belonging to a larger family of proteases known as the metzincin superfamily. These enzymes are capable of degrading extracellular matrix proteins which are bioactive molecules. They are known to be involved in the cleavage of cell surface receptors, the release of apoptotic ligands (such as the FAS ligand), and chemokine/cytokine inactivation. MMPs play a role in host defense and in tissue remodeling associated with various physiological or pathological processes such as morphogenesis, metastasis, angiogenesis, tissue repair, cirrhosis, and arthritis. MMPs that are expressed and/or upregulated on aberrant immune cells during immune disorders include, but not limited to, CD10, MMP19, MMP14, MMP24, ADAMTS4, ADAM28, ADAMTS17, ADAMTSL2, ADAMTS6, ADAM9, ADAMDEC1, ADAM33, ADAM10, ADAM17, ADAM8, and ADAM19.

One exemplary MMP is CD10 (also known as Neprilysin and Common Acute Lymphoblastic Leukemia Antigen, (CALLA)), a zinc-dependent membrane metallo-endopeptidase that cleaves peptides at the amino side of hydrophobic residues and inactivates several peptide hormones, including the B-chain of insulin, enkephalins, endothelin, atrial natriuretic peptide, substance P, and a chemotactic peptide. It also degrades the amyloid beta peptide whose abnormal folding and aggregation in neural tissue has been implicated as a cause of Alzheimer's disease. CD10 is expressed in a wide variety of tissues including lymphoid cells.

CD10 is an antigen that is a cancer cell surface marker in the diagnosis of human acute lymphocytic leukemia (ALL) and preB-ALL preB-AL, and has been used as a prognosis marker of this cancer, elevated levels of which on cancer cells suggest a poor clinical outcome. In addition, CD10 expression in low-density neutrophils has been described in the context of lupus (DOI: 10.1182/blood-2016-04-713206). Also, CD10 expression has been shown to be induced by pro-inflammatory signals like TNFα and IFNγ on mesenchymal stem cells (DOI: 10.1038/s41598-019-47391-2).

Another enzyme that can be targeted by the therapeutic compound according to the disclosure is a glucuronidase. A glucuronidase is an enzyme that hydrolyzes the glycosidic bond that links glucuronic acid to another molecules Glucuronidases are members of the glycosidase family of enzymes that catalyze breakdown of complex carbohydrates. For example, human β-glucuronidase is a type of glucuronidase (a member of glycosidase Family 2) that catalyzes hydrolysis of β-D-glucuronic acid residues from the non-reducing end of mucopolysaccharides such as heparan sulfate.

Various immune cells express glucuronidases during inflammation. Cytotoxic T cells, upon activation by specific target cells, secrete the content of cytoplasmic granules that contain β-glucuronidase. Macrophages and neutrophils are induced to secrete β-glucuronidase under inflammatory conditions. Autophagy/mitophagy impairment in inflammatory cells, inducing the activation of β-glucuronidase activity and resulting in mitochondrial dysfunction, is implicated in the induction of chronic inflammation.⁽⁴⁴⁻⁴⁶⁾

Another non-limiting enzyme that can be targeted by compounds according to the disclosure is a sulfatase. Sulfatases are enzymes of the esterase class that catalyze the hydrolysis of sulfate esters, which are found on steroids, carbohydrates and proteins.

Various receptors found on the surface of aberrant immune cells may also be targeted by compounds according to the disclosure. Some representative and useful receptors include, but are not limited to IL2RA, IL4R, IL6R, IL7R, IL9R, IL21R, IL23R, TACI, AICDA, FcRL4, FcγRIIB, CX3CR, AC073136.1 (nuclear export receptor for tRNAs), CASR, NR4A2, ADGRG1, GRM4, CXCR2, GRASP, RGMA, SORCS2, GPRC5A, PTGFR, TNFRSF10C, GRM2, EPHA2, GPR82, CCR6, GPR52, MSR1, CSF1R, HCAR2, MILR1, REEP2, TRAJ35, FZD9, F2R, GPR87, TRAJ49, RTP5, FCRL4, PTPRVP, OR2T10, HTR2B, OR2G6, LRP1, GPR176, PTPRF, CCR9, CELSR1, GPR3, RARRES2, HCAR3, TNFRSF18, PTPRM, NPR1, TIGIT, TNFRSF4, GABARAPLI, SPSB3, FCRL2, CCR12P, FFAR2, RARRES3, CD1C, CD1D, CD14, CD22, CD81, CD96, CD209, CD247, CD276, and CD302.

### Identifying/Monitoring Aberrant Immune Cells

Identification and monitoring aberrant immune cells to diagnose the presence of an immune-related disorder can be accomplished by monitoring the level of expression of certain enzymes in the blood or serum of a subject. Once the presence of the protein is detected or the over-expressed level of the protein determined, the disorder can be treated with a compound comprising a therapeutic moiety according to the disclosure.

Measurement of the levels of an aberrant immune cell-expressed enzyme or receptor in a subject or sample taken from the subject can be carried out in a variety of methods known in the art. In many cases, levels of gene expression correlate with levels of enzyme or receptor. In such cases, aberrant immune cell-related protein expression can be monitored by quantitating mRNA transcribed from a product gene in the immune cell isolated from the sample. This can be accomplished by Northern hybridization (see, Sambrook et al., Molecular Cloning: A Laboratory Manual, pp. 7.3-7.57 (Cold Spring Harbor Laboratory Press, 1989).

An MMP protein encoded by a particular product gene can be quantitated either by assaying for the biological activity of the protein or by employing assays that are independent of such activity, such as, but not limited to, Western blotting, ELISA, or radioimmunoassay using MMP-specific antibodies (see, Sambrook et al., ibid., pp. 18.1-18.88).

Gene expression can also be monitored by quantitative polymerase chain reaction (qPCR) (also known as real time PCR). In conjunction with reverse transcription, which converts mRNA to complementary DNA (cDNA), qPCR is used to amplify and quantitate cDNA specific to the expression of a gene of interest. Two exemplary common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

Another monitoring method uses mass spectrometry (MS). In brief, PBMCs isolated from a patient sample are analyzed with MS-based proteomics.

(doi: 10.1016/j.cbpa.2008.07.024). Peptides derived from the protein are quantified to indicate disease activity.

Flow cytometry using labeled specific anti-aberrant immune cell-expressed protein, immunoglobulins and activity-based probes that are fluorogenic or chromogenic can also be used. In brief, a patient sample (whole blood or serum) can be labeled with fluorescent antibody directed to one or more enzymes or receptors (e.g., anti-CD10 and anti-CD19 antibodies) or directed to one or more enzymes and quantified with a flow cytometer. For example, Elevated counts of the CD10+CD19+ population indicate higher disease activity.

### Treatment for Immune-Related Disorders

Immune-related disorders associated with aberrant immune cells expressing an enzyme or receptor, e.g., an MMP, glucuronidase, or sulfatase, can be treated by directing a therapeutic compound to the immune cell. Such a compound comprises a “Bait” or specific enzyme- or receptor-targeting ligand that binds the enzyme or receptor, and a Payload which reduces or inhibits the aberrant activity of, or which kills, the aberrant immune cell.

### A. Baits

Baits correspond to one or more ligands that may each bind a specific protein, such as an enzyme or receptor expressed on or within an aberrant immune cell. The Bait is a molecule comprising one or more Subunits, e.g., from about one to about five Subunits, which can be one or multiples of the same or a combination of different small organic molecules such as, but not limited to, amino acids, sugars, and/or lipids, including, but not limited to, those existing in nature, and an optional “Cap” terminating the Subunits and comprised of a small organic molecule such as, but not limited to, carboxylic acids, ureas, carbamates, and sulfonates, including, but not limited to, those existing in nature This component may optimize the therapeutic compound to achieve aberrant immune cell-selective targeting and accumulation by serving, for example, as a substrate for the targeted enzyme(s) or receptor(s) present on or in the targeted aberrant immune cells.

In some compounds according to the disclosure, the Bait does not affect the therapeutic activity of the Payload when attached thereto. However, in some cases, the Bait may serve to stabilize the compound, and may assist in inhibiting the activity of the Payload, e.g., by restricting cell permeability or by electrostatic or steric interference with regions on the target of the Payload, for example. Thus, under certain circumstances, the activity of the Payload moiety in the compound may be inhibited or inactive until the Bait moiety is released. Under these circumstances, cleavage of the Bait moiety and release of the Payload occurs when certain disease factors are present, as they may be in the aberrant immune cells, thus sparing unaffected cells. As used herein, the term “disease factors” encompasses compounds and molecules present in the environment of the aberrant immune cells and which are associated with the immune-related disorder, and may be synthesized or elicited by the aberrant immune cells. Representative, nonlimiting disease factors include small molecules (chemical compounds) or macromolecules that circulate in plasma or lymph of a subject with the immune-related disorder, but not in healthy subjects or subjects that have their diseases in remission. The presence of a disease factor may result in the production or activation of the enzyme or receptor that causes or results in the cleavage of the Bait from the compound.

The Bait may also be in the form of a proBait that when metabolized by the host or subject that potentially harbors an aberrant immune cell and may have an autoimmune disorder, becomes a Bait.

The Bait moiety of the compounds of the disclosure includes binding agents such as substrates of or immunoglobulins specific for, the targeted enzyme(s) or receptor(s).

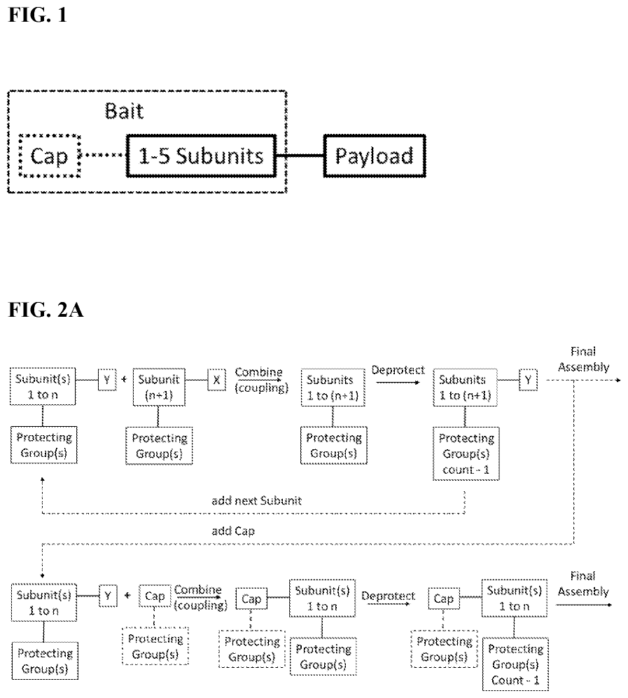

Natural as well as non-native or artificial substrates of the targeted enzyme or receptor are used as a Bait to bind to the aberrant immune cell expressing the targeted enzyme or receptor. The substrate can be an oligomer comprising from about one to about five Subunits with an optional “Cap” and is directly or indirectly attached to the Payload (FIG. 1).

Useful representative Caps include:

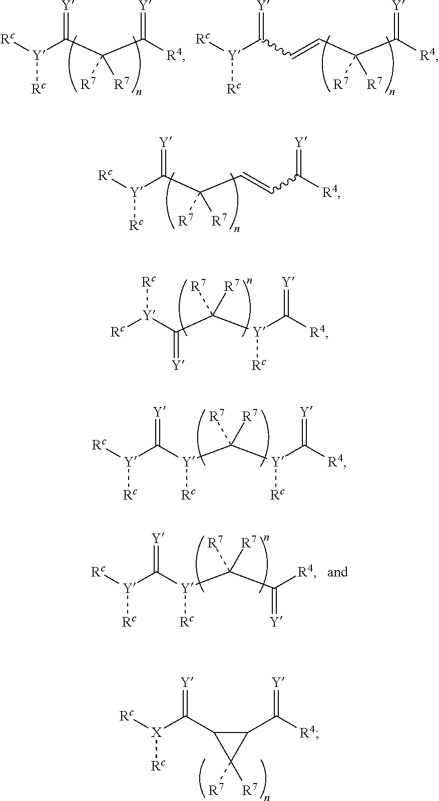

wherein R⁴ is independently, at each occurrence, selected from the group consisting of a bond to any R⁸ or any R⁶ position in a Subunit.

Useful representative Subunits of the substrate include, independently at each occurrence:

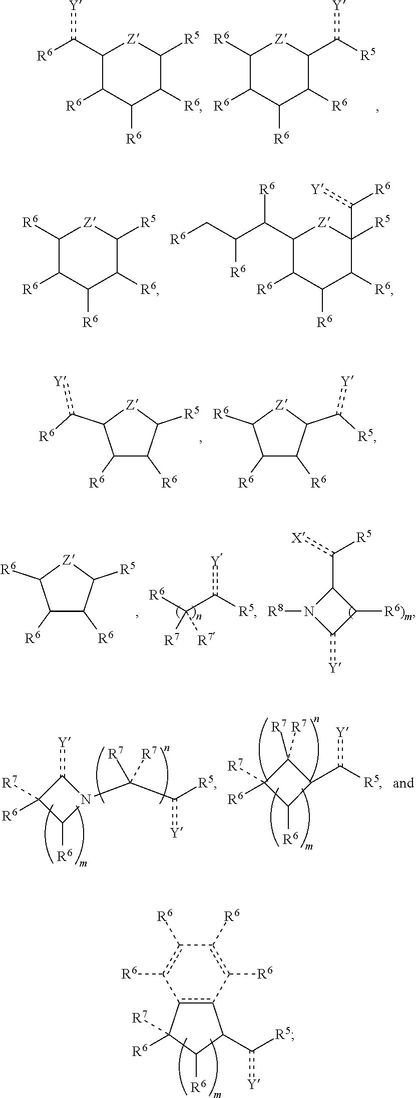

wherein:
--- is an optional single bond;
=== is an optional single or double bond;
Z′ is independently, at each occurrence, selected from the group consisting of CH₂, NH, O, and S;
X′ and Y′ are independently, at each occurrence, selected from the group consisting of O, NH, and S;
   R⁵ is independently, at each occurrence, selected from the group consisting of a bond to an R⁶ in another Subunit, a bond to an R⁸ in another Subunit, or a bond to the Payload;
R⁶ is independently, at each occurrence, selected from the group consisting of H, —R^{D}, —R^{A}—R⁵ when R⁵ is in another Subunit, —R^{A}—R⁴ when R⁴ is in a Cap, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with 1-4 four halo(s);
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with a substituent selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, C₃₋₄ allyl, and a combination thereof;
R⁸ is independently, at each occurrence, a bond to R⁵ in another Subunit, or a bond to an R⁴ in the Cap;
m is 0, 1, 2, 3, or 4; and
n is 0, 1, 2, or 3.

Specific nonlimiting examples of substrates include, but are not limited to, N-succinyl-alanine-alanine-phenylalanine (SAAP) (SEQ ID NO: 1), N-glutaryl-alanine-alanine-phenylalanine (GAAP) (SEQ ID NO:2), N-succinyl-alanine-alanine-leucine (SAAL)- (SEQ ID NO:3), and N-glutaryl-alanine-alanine-leucine (GAAL SEQ ID NO:4).

Other specific, nonlimiting examples of substrates comprise:

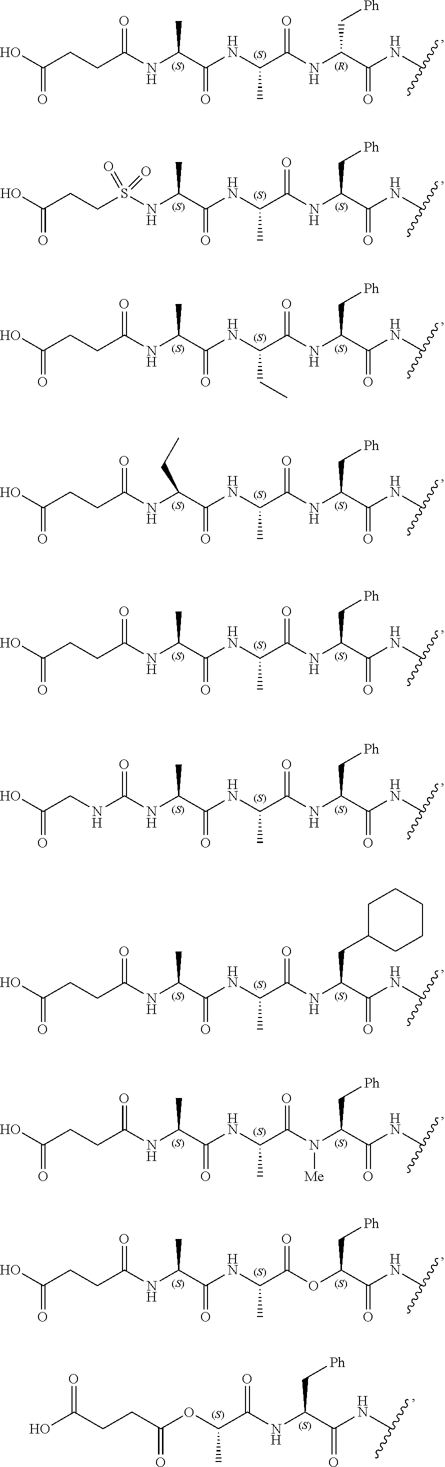

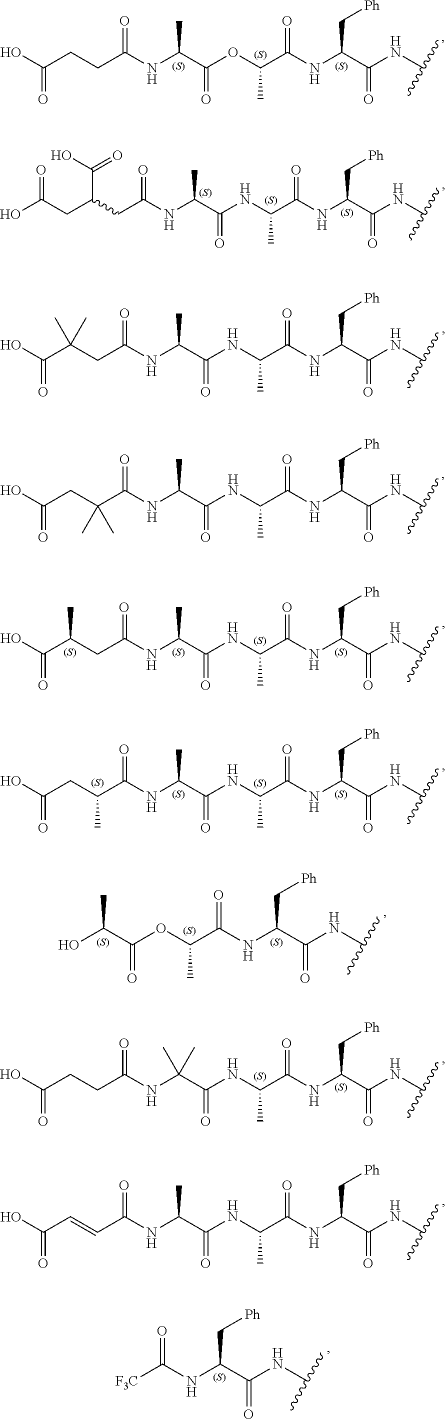

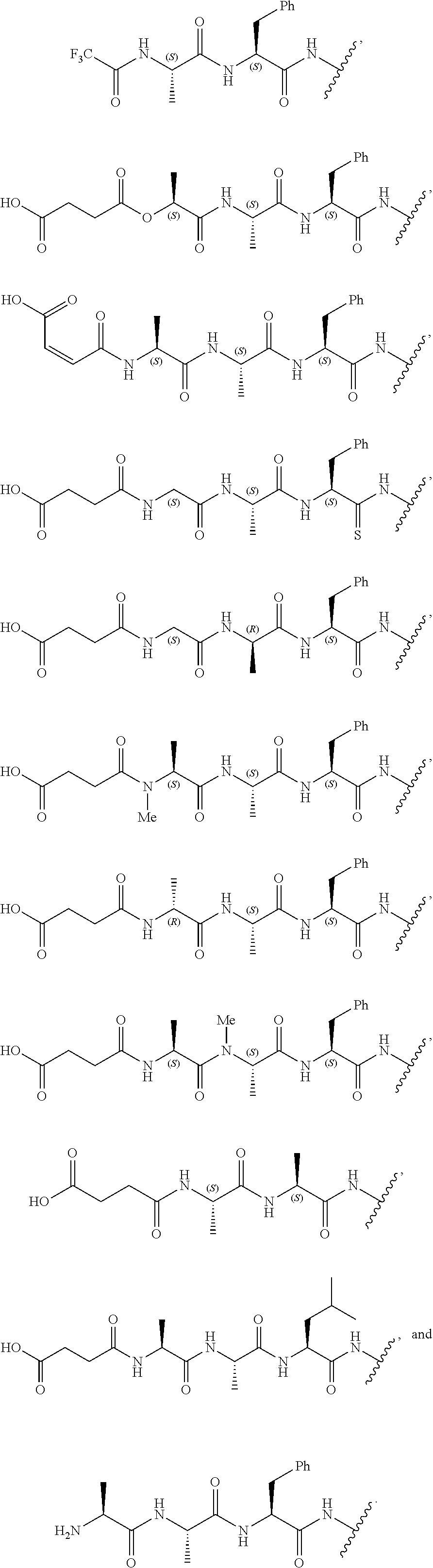

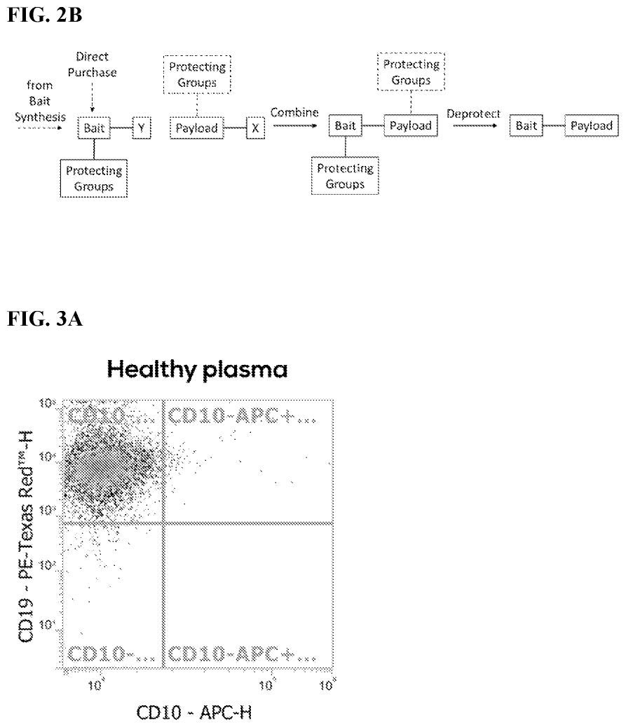

Substrate (i.e., Bait) components (i.e., Subunits and Cap,) may be obtained commercially or synthesized by any method known by those with skill in the art. For Bait components that are not commercially available as ready units for use in the final assembly (e.g Fmoc-Gly-Tyr(tBu)-OH a.k.a. Pubchem ID=329767849, one non-limiting example of a commercially available protected Subunit) Substrates are individually synthesized as per the general scheme shown in FIG. 2A as one example. In FIG. 2A all boxed entities represent chemical matter, either a whole molecule or where joined by a line represent a portion of a molecule connected by one or more chemical bonds to the other portion(s) as indicated by the connecting line; arrows indicate actions such as synthetic step(s), solid lines for both boxes and arrows represent required steps and molecules or portions thereof; and dashed lines for boxes and arrows represent steps and molecules or portions thereof that may be conditional.

Within FIG. 2A individual “Combine” steps involve coupling amino acid and/or sugar components together via standard methods (e.g., Jaradat (2017) Amino Acids. 50 (1): 39-68) and/or oligosaccharide synthesis (e.g., Levy et al., The Organic Chemistry of Sugar; Taylor & Francis: 2006) depending on the composition of a given Substrate. When Substrates are built up from commercially available units, this is performed in a sequential manner of coupling and deprotecting as generically outlined in FIG. 2A Caps are added via standard relevant methods such as acylation. For a Substrate to be ready to be attached to a Payload directly, a single R5 position that is the point of attachment to Payload is left un-protected (labeled as “Y” in FIG. 2A) while all other aliphatic amines and protic functionality with a pKa<20 in DMSO are protected via standard means (see, e.g., Wuts and Green; Greene's Protective Groups in Organic Synthesis, Fourth Ed.; Joh n Wiley & Sons, Inc.: 2007).

The final assembly (FIG. 2B) is carried out by coupling the appropriately protected Bait, either from the Bait synthesis as described supra, or commercially obtained, and with or without protecting groups as necessary, described supra. This is done with an appropriate coupling reaction used for the exposed functionality on both the Payload and Bait (e.g., “Bait” presenting a “Y”, a.k.a. R⁵, with a carboxyl group may be coupled to a “Payload” presenting an “X” that was an aliphatic amine with N,N′-Dicyclohexylcarbodiimide.) All residual protecting groups are then removed from the resulting assembled compound to yield the active final “Bait”-“Payload” assembly.

These and other non-native MMP substrate molecules can be used as part of next-generation therapeutics of immune-related diseases which also comprise prodrug conjugates that modulate the enzymatic function. For example, these MMP substrates can be used to mask the binding region of therapeutic antibodies and antibody-related compositions useful in treating in immune-related diseases, allowing these antibody-related compositions to be activated only during active diseases. Examples of such antibody-related compositions include anti-TNF antibodies (and their biosimilars), e.g., Infliximab, Etanercept, Adalimumab, Golimumab, Certolizumab pegol; anti-CD80 and anti CD86 antibodies (e.g., Abatacept), anti-IL-6 receptor antibodies (e.g., Tocilizumab), anti-Interleukin-1 type I receptor (IL-1RI) antibodies (e.g., Anakinra), anti-IL-12/23 antibodies (e.g., Ustekinumab), anti-CD20 antibodies (e.g., Rituximab), anti-interleukin-17 antibodies (e.g., Secukinumab), anti-CD19 antibodies, and anti-CD22 antibodies.

The Bait alternatively an immunoglobulin ligand specific for the target enzyme or receptor include antibodies and binding portions thereof, bispecific antibodies, single domain immunoglobulins, camelids, and chimeric antigen receptors (CAR) CAR T cells.

The antibodies can be in the form of full length antibodies, or in the form of fragments of antibodies, e.g., Fab, F(ab′)2, Fd, Fv, dAb, and scFv fragments. Additional forms include a protein that includes a single variable domain, e.g., a camelid or camelized domain. (see, e.g., U.S. 2005-0079574 and Davies et al, (1996) Protein Eng. 9(6):531-7). The antibody can be a human, humanized, CDR-grafted, chimeric, mutated, affinity matured, deimmunized, synthetic or otherwise in vitro-generated antibody, and combinations thereof. The antibody can include an antigen-binding fragment (e.g., a Fab, F(ab′)2, Fv or a single chain Fv fragment), a heavy chain constant region chosen from, e.g., IgG1, IgG2, IgG13, IgG4, IgM, IgGA1, IgA2, IgD, and IgE, and/or a light chain constant region chosen from, e.g., kappa or lambda or kappa.

Such antibodies can be made by any method known in the art. For example, an MMP, glucuronidase, or sulfatase, or a peptide thereof can be used as an antigen to produce an antibody in a non-human animal. Alternatively, a monoclonal antibody can be prepared from the non-human animal, and then modified, e.g., humanized or deimmunized (see, e.g., U.S. Pat. No. 5,225,539). The antibody can include a human Fc region, e.g., a wild-type Fc region or an Fc region that includes one or more alterations, e.g., Fc receptor binding and complement activation. For example, antibodies can have mutations such as those described in U.S. Pat. Nos. 5,624,821 and 5,648,260. Antibodies can also have mutations that stabilize the disulfide bond between the two heavy chains of an immunoglobulin, such as mutations in the hinge region of IgG14, as disclosed in the art (e.g., Angal et. al, (1993) Mol. Immunol. 30:105-08. The antibody can be modified to have an altered glycosylation pattern (i.e., altered from the original or native glycosylation pattern) (see. e.g., WO 87/05330, and Aplin and Wriston (1981) CRC Crit. Rev. Biochem. 22:259-306). Removal of any carbohydrate moieties present on the antibodies can be accomplished chemically or enzymatically as described in the art (Hakimuddin et al, (1987) Arch. Biochem. Biophys. 259:52; Edge et al. (1981) Anal. Biochem. 118:131; and Thotakura et al. (1987) Meth. Enzymol. 138:350). Recombinant antibodies can be made by preparing and expressing synthetic genes that encode the recited amino acid sequences or by mutating human germline genes to provide a gene that encodes the recited amino acid sequences which recognizes CD10. Another exemplary method includes screening protein expression libraries, e.g. phage or ribosome display libraries (see, e.g., U.S. Pat. Nos. 5,223,409; 5,658,727; 5,667,988; and 5,885,793. Smith (1985) Science 228:1315-1317; WO 92/18619; WO 91/17271; WO 92/20791; and WO 90/02809).

The binding agent can also be a chimeric antibody receptor (CAR) targeting the enzyme or receptor, or can be a CAR on a T cell (CAR T) such as genetically engineered T-lymphocytes (T cells), naive T cells, memory T cells (for example, central memory T cells (TCM), effector memory cells (TEM)), natural killer cells (NK cells), and macrophages Capable of giving rise to therapeutically relevant progeny. The CAR T cells can be autologous cells. Such modified immune cells (e.g., comprising a TCR and/or CAR) may be produced by stably transfecting host cells with an expression vector including a nucleic acid of the present disclosure. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing an immune receptor may be expanded ex vivo. (see, e.g., Rafiq (2020) Nat. Rev. Clin. Oncol. 17:147-167; Lapot (2003) Blood 102:2004-2013; Levine (2006) Proc. Natl. Acad. Sci. U.S.A. 103:17372-17377.

The ligand can also be labeled such that the aberrant immune cell to which it binds can be identified. Such labels include radionuclides, colorimetric, fluorescent, biotin/avidin, or affinity-based labels that are attached to the ligand by any method known in the art.

### Payloads

The Bait is linked or conjugated to a Payload which comprises a therapeutic molecule that inhibits or reduces the pathological activity of the aberrant immune cell, or which kills it. Such a therapeutic molecule comprises a small molecule inhibitor or cytotoxic agent, an immunoglobulin specific for a target other than the enzyme or receptor to which the Bait specifically binds, an antibody-drug conjugate, and an inhibitor of an enzyme needed for cell metabolism. Such Payloads can also be a label or can be labeled.

Therapeutic, small molecule Payloads include cytotoxic agents, inhibitors an immune cell enzyme, or inhibitors of an immune cell receptor. Useful small molecule Payloads include, but are not limited to, inosine-5′-monophosphate dehydrogenase (IMPDH)-inhibiting small molecule compounds that are proliferative disease-modifying, immune modulatory, and are selective for the targeting-enzyme/receptor-expressing B cells, Other useful Payloads include, but are not limited to, inhibitors of a glucocorticoid receptor, e.g., Prednisone, Budesonide, Prednisolone, Dexamethasone, and Hydrocortisone, inhibitors of a Janus kinase, e.g., Tofacitinib (Xeljanz), Baricitinib, Oclacitinib, Ruxolitinib, Filgotinib, Decemotinib, Upadacitinib, and Peficitinib, inhibitors of calcineurin, e.g., Cyclosporine, Voclosporin, Tacrolimus, and Pimecrolimus, inhibitors of mammalian target of rapamycin (mTOR), e.g., Rapamycin and Everolimus, and inhibitors of dihydrofolate reductase (DHFR). Other useful Payloads include, but are not limited to, inhibitors of an enzyme involved in purine synthesis and metabolism, e.g., inhibitors of adenylosuccinate synthase, adenylosuccinate lyase, inosine-monophosphate dehydrogenase, GMP synthase, and glutamine-phosphoribosyl pyrophosphate amidotransferase (GPAT), inhibitors of dihydroorotate dehydrogenase (DHODH), e.g., leflunomide or brequinar. The Payload may also be a folic acid analog such as, but not limited to, methotrexate, or a purine analog such as, but not limited to, azathioprine ormercaptopurine. Other useful Payloads also include, but are not limited to, modulators of the sphingosine-1-phosphate receptor (SIP) receptor, e.g., Fingolimod, Ozanimod, and Ponesimod. The small molecule inhibitor is linked to the anti-MMP Bait directly or via proper linker chemistry.

Immunoglobulins include those that are specific for a target other than the protein to which the Bait binds, e.g., an MMP, glucuronidase, or sulfatase. Such immunoglobulins include any antibody or binding fragment thereof, camelid, single domain immunoglobulin, or CAR. The Bait is linked directly or via linker to the immunoglobulin Payload by any method known in the art.

Antibody-drug conjugates (ADCs) comprise any antibody specific for a target on the immune cell other than the MMP to which the Bait is directed, linked directly or via linker to a cytotoxic agent. Some useful, nonlimiting therapeutic molecules to which Payload ligands (such as antibodies or binding fragments thereof), can be conjugated include inosine-5′-monophosphate dehydrogenase (IMPDH)-inhibiting small molecule compounds that are proliferative disease-modifying, immune modulatory, and are selective for CD10-expressing B cells, Other useful Payloads include, but are not limited to, inhibitors of a glucocorticoid receptor, e.g., Prednisone, Budesonide, Prednisolone, Dexamethasone, and Hydrocortisone, inhibitors of a Janus kinase, e.g., Tofacitinib (Xeljanz), Baricitinib, Oclacitinib, Ruxolitinib, Filgotinib, Decernotinib, Upadacitinib, and Peficitinib, inhibitors of calcineurin, e.g., Cyclosporine, Voclosporin, Tacrolimus, and Pimecrolimus, inhibitors of mammalian target of rapamycin (mTOR), e.g., Rapamycin and Everolimus, and inhibitors of dihydrofolate reductase (DHFR). Other useful Payloads include, but are not limited to, inhibitors of an enzyme involved in purine synthesis and metabolism, e.g., inhibitors of adenylosuccinate synthase, adenylosuccinate lyase, inosine-monophosphate dehydrogenase, GMP synthase, and glutamine-phosphoribosyl pyrophosphate amidotransferase (GPAT), inhibitors of dihydroorotate dehydrogenase (DHODH), e.g., leflunomide or brequinar. The Payload may also be a folic acid analog such as, but not limited to, methotrexate, or a purine analog such as, but not limited to, azathioprine or merCaptopurinene. Other useful Payloads also include, but are not limited to, modulators of the sphingosine-1-phosphate receptor (SIP) receptor, e.g., Fingolimod, Ozanimod, and Ponesimod.

### Immune-Related Disorders

Immune-related disorders treatable with aberrant immune cell-targeted compounds according to the disclosure include, but are not limited to, systemic lupus erythematosus (SLE), rheumatoid arthritis (RA), multiple sclerosis (MS), pemphigus vulgaris, bullous pemphigoid, scleroderma, myasthenia gravis, psoriasis, autoimmune myositis, as well as infection by SARS-CoV2, in addition to. Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease, (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

### Pharmaceutical Formulations

The presence of an aberrant immune cell can be identified and monitored via the use of a composition formulated to bind to and label a protein being expressed or over-expressed by the aberrant immune cell. Such a formulation includes a labeled ligand Bait which specifically binds to the particular aberrant immune cell protein (receptor or enzyme) and a carrier which does not affect the ability of the ligand to bind to its target. This formulation can be used to assay a blood or serum sample taken from the subject.in an amount sufficient to detect the protein on aberrant immune cells present in the subject or sample. The ligand may be labeled with a fluorescent, colorimetric, isotopic, or other markers for ease of detection. Methods of protein labeling are well known in the art.

In addition, the compound according to the disclosure can be formulated for in vivo delivery to, and treatment of, a subject suffering from, or potentially having, or at risk for, an immune-related disorder. A therapeutically effective amount of the compound is formulated with a pharmaceutically acceptable carrier that does not affect the ability of the Bait moiety of the compound to bind a specific protein target and the ability of the Payload moiety of the composition to inhibit or kill the aberrant immune cell.

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe Handbook of Pharmaceutical. Excipients (6^{th} ed., Rowe et al., eds. (2009) Pharmaceutical Press Grayslake, IL).

As used herein, “pharmaceutically acceptable carrier” includes any and all solvents, dispersion media, coatings, antibacterial and antifungal agents, isotonic and absorption delaying agents; and the like that are physiologically compatible with the inhibitor and which does not reduce its inhibitory activity. The composition can include a pharmaceutically acceptable salt, e.g., an acid addition salt or a base addition salt of the compound which is the inhibitor (see e.g., Berge et al. (1977) J. Pharm. Sci. 66:1-19).

The pharmaceutical compositions can be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application.

For example, such formulations can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Such parenterally administered formulations are prepared as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Injectable dosage forms may be sterilized in a pharmaceutically acceptable fashion, for example by steam sterilization of an aqueous solution sealed in a vial under an inert gas atmosphere at 120° C. for about 15 minutes to 20 minutes, or by sterile filtration of a solution through a 0.2 μM or smaller pore-size filter, optionally followed by a lyophilization step, or by irradiation of a composition containing an inhibitor of the present disclosure by means of emissions from a radionuclide source.

A therapeutically effective dosage of the formulation according to the disclosure depends on the disorder treated and may vary from patient to patient, and factors such as the age and physical size of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may be determined in view of the age, sex, weight, and genetic background of the subject, and can vary or change as a function of time and severity of the immune disorder. A subject can also be prophylactically treated with the formulation so as to avoid or delay development of an immune-related disorder.

A dosage from about 0.1 μg/kg to about 1000 mg/kg, or from about 1 mg/kg to about 100 mg/kg compound may be suitable. For example, for the treatment of immune-related disorders, a suitable dosage level is about 0.01 μg/kg to about 1000 mg/kg of the compound. Some useful unit dosage forms contain from 1 μg/kg to about 500 mg/kg, from about 10 μg/kg to about 100 mg/kg, from about 100 μg/kg to about 500 mg/kg, about 0.1 μg/kg to about 10 μg, about 100 μg/kg to about 1 mg/kg, about 10 mg/kg, about 100 mg/kg, about 500 mg/kg, about 1000 mg/kg, or from about 0.001 mg/kg to about 250 mg/kg per day. The formulation maybe administered via bolus and/or a regimen of from about 1 times per day to about 4 times per day. Alternatively, it may be administered continuously for a period of time determined by a physician.

For preparing solid compositions such as tablets, the compound is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a pharmaceutically acceptable salt thereof.

These formulations may be homogeneous, i.e., the compound according to the disclosure is dispersed evenly throughout the composition so that the composition may be readily subdivided into equally effective unit dosage forms such as tablets, pills and Capsules. This solid formulation composition is then subdivided into unit dosage forms of the type described above containing from about 0.01 μg to about 1000 mg of the compound. Some useful unit dosage forms contain from 1 μg to about 500 mg, from 10 μg to about 100 mg, from about 100 μg to about 500 mg, for example, about 0.1 μg to about 1.0 μg to about 10 μg, about 100 μg, about 1 mg, about 10 mg, about 100 mg, about 500 mg, or about 1000 mg the compound. The tablets or pills comprising the compound according to the disclosure can be coated or otherwise compounded to provide a dosage form affording the advantage of prolonged action. The liquid forms in which the compound of the present disclosure may be incorporated for administration orally or by injection include aqueous solutions, suitably flavored syrups, aqueous or oil suspensions, and flavored emulsions with edible oils as well as elixirs and similar pharmaceutical vehicles.

The compound-containing formulations according to the disclosure may additionally comprise another therapeutically effective agent that is different than the aberrant immune cell targeted and binding compound described above. Nonlimiting, exemplary therapeutic agents other immune cell inhibitors and/or other therapeutic agents that treat the immune disorder. Such therapeutic agents include, but are not limited to, glucocorticoids, cyclosporin A, azathioprine, mycophenolate mofetil, tacrolimus, and sirolimus. Alternatively, when treating a subject with an aberrant immune cell-directed formulation according to the disclosure, treatment may also include administering another formulation comprising a therapeutic agent effective in treating the immune-related disorder, the agent being is different than the compound in the aberrant immune cell-targeted formulation set forth above.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Confirmation of CD10 Induction by Flow Cytometry

2×10⁵ primary CD19+ B cells isolated from a healthy donor blood were stimulated with 10% v/v_plasma from either a healthy donor, a pemphigus vulgaris (PV) donor, or a systemic lupus erythematosus (SLE) donor for 24 hours at 37 C, 5% CO₂. Cells were then labeled with both anti-CD10 antibody (clone HI10a, APC, Biolegend, CA) and anti-CD19 antibody (5 μl/million cells in 100 μl volume, clone HIB, PE-CF594, Biolegend, CA), and analyzed by flow cytometry using an Attune NxT Flow Cytometer (ThermoFisher).

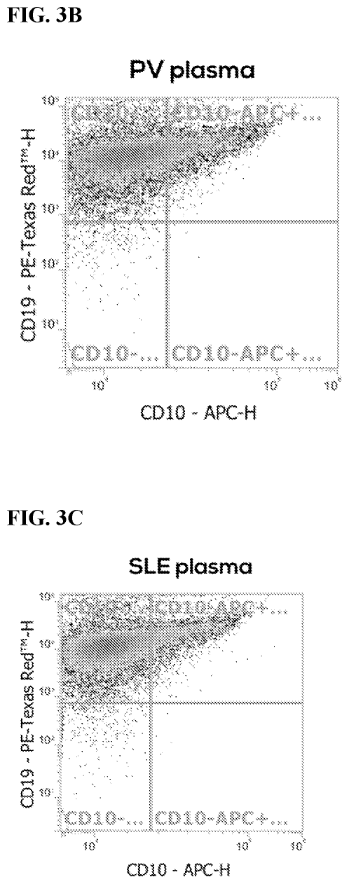

Among CD19+ B cell populations, about 30% were induced to express high level of CD10 as measured by allophycocyanin (APC)-labelled anti-CD10 antibody when PV (FIG. 3B) or SLE (FIG. 3C) plasma is present, whereas healthy donor's plasma did not trigger the induction of CD10 (FIG. 3A). Thus, CD10, and similar MMPs are useful diagnostic and prognostic markers for pemphigus vulgaris PV, SLE, and similar immune-related disorders.

### Example 2

### Proliferation Assay

0.5×10⁶ primary cells (purified CD19+ primary B cells, Precision for Medicine, MD) or 0.5×10⁶ CD3+ primary T cells (Precision for Medicine, MD) from a healthy donor were assayed for their rate of proliferation via the BrdU incorporation method (Roche kit 11647229001), following vendor protocols. In brief, BrdU was added to cell culture for 2 hours-12 hours. The cells were dried with hot air ventilation and fixed with saline and ethanol. The incorporated BrdU was measured with an ELISA assay with an anti BrdU antibody (Roche).

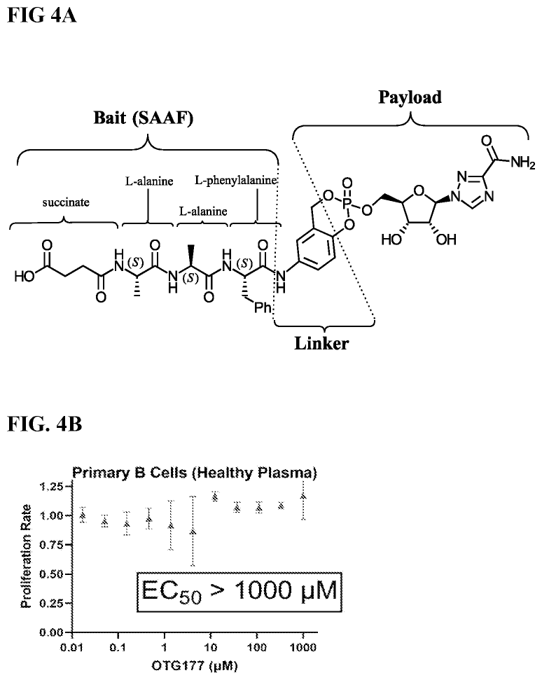

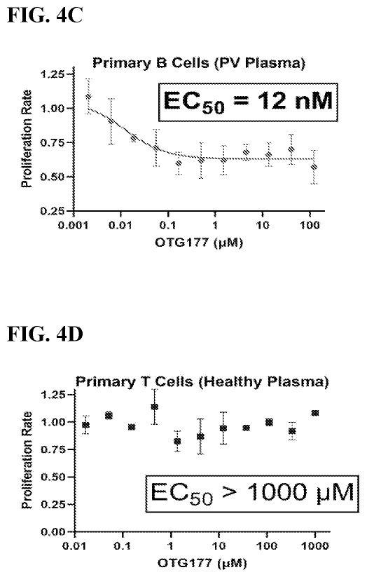

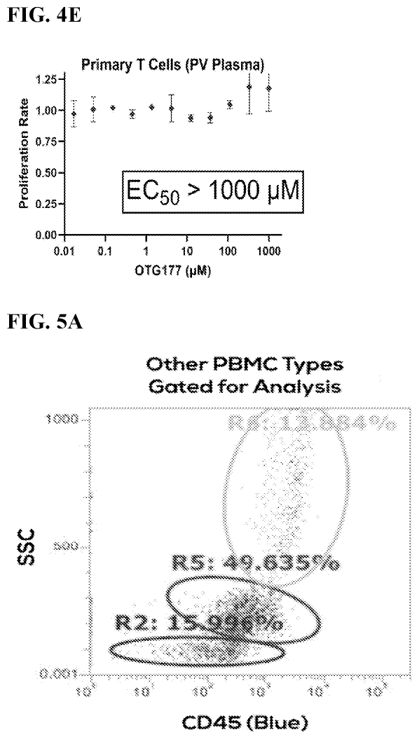

A representative compound according to the disclosure, OTG177 (FIG. 4), was constructed according to the synthetic scheme shown in FIGS. 2A and 2B using the Bait CD10 substrate succinyl-Ala-Ala-Phe- and an antiproliferative Payload RMP. This compound was tested in this proliferation assay as follows: negative selected lymphocytes (either B or T cells) were incubated with different concentration of OTG177 up to 1000 μM with 10% plasma from either a healthy donor or a flaring Pemphigus Vulgaris donor. All conditions had 30 units/ml of heparin to prevent clotting of the plasma. Cells were incubated for 72 hours at 37° C. 5% CO₂ and a BrdU incorporation assay was performed with kit (Roche) as described before.

As shown in FIG. 4B, OTG177 did not exhibit detectable antiproliferative activity in B cells stimulated by a healthy donor's plasma, but exhibited strong antiproliferative activity in B cells stimulated by a flaring pemphigus vulgaris donor's plasma (FIG. 4C). Because T cells lack CD10 expression, OTG177 did not exhibit antiproliferative activity in T cells stimulated in either a healthy donor's plasma (FIG. 4D) or a flaring pemphigus vulgaris donor's plasma (FIG. 4E).

These results demonstrate that CD10 induction observed in Example 1 activates therapeutic molecules according to the disclosure in a disease-state-specific manner.

### Example 3

### Mixed-Cell Assay

To determine if the selective antiproliferative activity of OTG177 affects immune cells in the blood in addition to B cells, a mixed cell assay was performed as follows:

0.5×10⁶ primary cells (purified CD19+ primary B cells (Precision for Medicine, MD) or 0.5×10⁶ CD3+ primary T cells (Precision for Medicine, MD Primary PBMCs (Precision for Medicine, MD) from a healthy donor were stimulated with 10% v/v plasma from either a healthy donor, a pemphigus vulgaris (PV) donor, or a systemic lupus erythematosus (SLE) donor for 24 hours at 37 C, 5% CO₂. Compound OTG177 was added at 1 μM, 10 μM and 50 μM concentrations with DMSO as a mock treatment. After 24 hours-72 hours of treatment, cells were labeled with anti-CD45 antibody (5 μl/million cells in 100 μl volume, clone HI30, BUV395, Biolegend, CA), anti-CD19 antibody (5 μl per million cells in 100 μl volume, Clone HIB, PE-CF594, Biolegend, CA) anti-CD3 antibody (5 μl/million cells in 100 μl volume, clone SK7, FITC, Biolegend, CA). Propidium (1 μg/ml) was used to select cells based on their viability. The cells were analyzed by multi-colored flow cytometry using an Attune NxT Flow Cytometer (ThermoFisher).

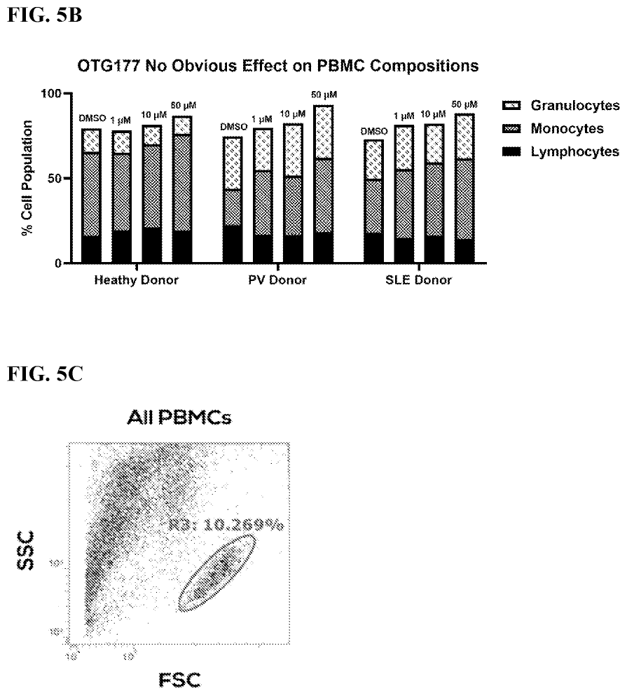

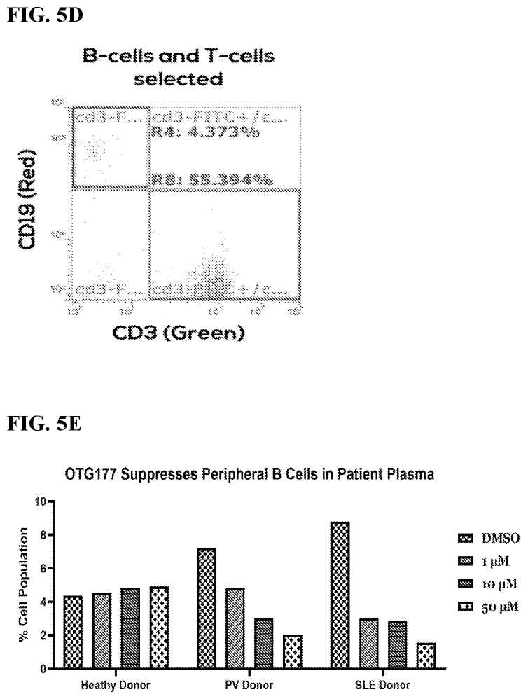

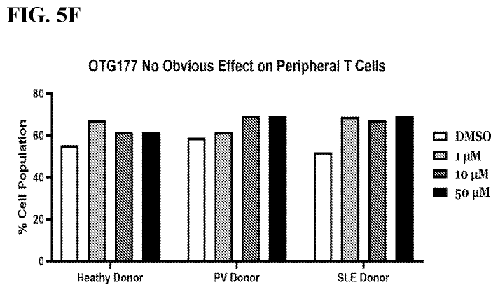

Overall peripheral blood mononuclear cell (PBMC) composition was evaluated by gating side scattering (SSC) versus CD45, a pan leukocyte marker (FIG. 5A). This gating strategy can differentiate different leukocyte populations based on their scattering profiles.

As shown in FIG. 5B, PBMC composition was not affected by up to 50 μM of OTG177. However, stimulation with patient (PV and SLE plasma, but not healthy plasma, increased the number of granulocytes respectively; and decreased by 28 and 18% the number of monocytes, respectively. However, the percentage of lymphocytes remained stable.

In addition, the effect of OTG177 on B and T lymphocytes was examined by first gating all PBMCs by forward scattering (FSC) versus side scattering (SSC) and isolating the low SSC and high FSC population (FIG. 5C), gating on this selection, the populations of segregated based on their staining to CD19 (red) and CD3 antibodies (green) (FIG. 5D).

As shown in FIG. 5E, B cells were not affected by OTG177 when stimulated with healthy plasma. However, B cells proliferated when stimulated with patient PV and SLE plasma, and OTG177 exhibited dose-dependent suppression of B cell proliferation (in the 1 μM to 50 μM range). In contrast, T cells were not affected by OTG177, even up to the 50 μM concentration, in presence of all plasma (FIG. 5F).

These results indicate that the cell type- and disease state-selective anti-proliferative agent OTG177 is highly selective in the peripheral blood settings. OTG177 exemplifies that when a disease biomarker is induced to appear or increase on the surface or inside relevant cell populations during the immune disease process, can be exploited to prepare highly targeted therapeutic molecules that modulate relevant immune cell populations selectively in immune-related disease conditions.

### Example 4

### Synthesis of 1-[(2R,3R,4S,5R)-5-[[6-[[(2S)-2,6-diaminohexanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide Using Scheme 1

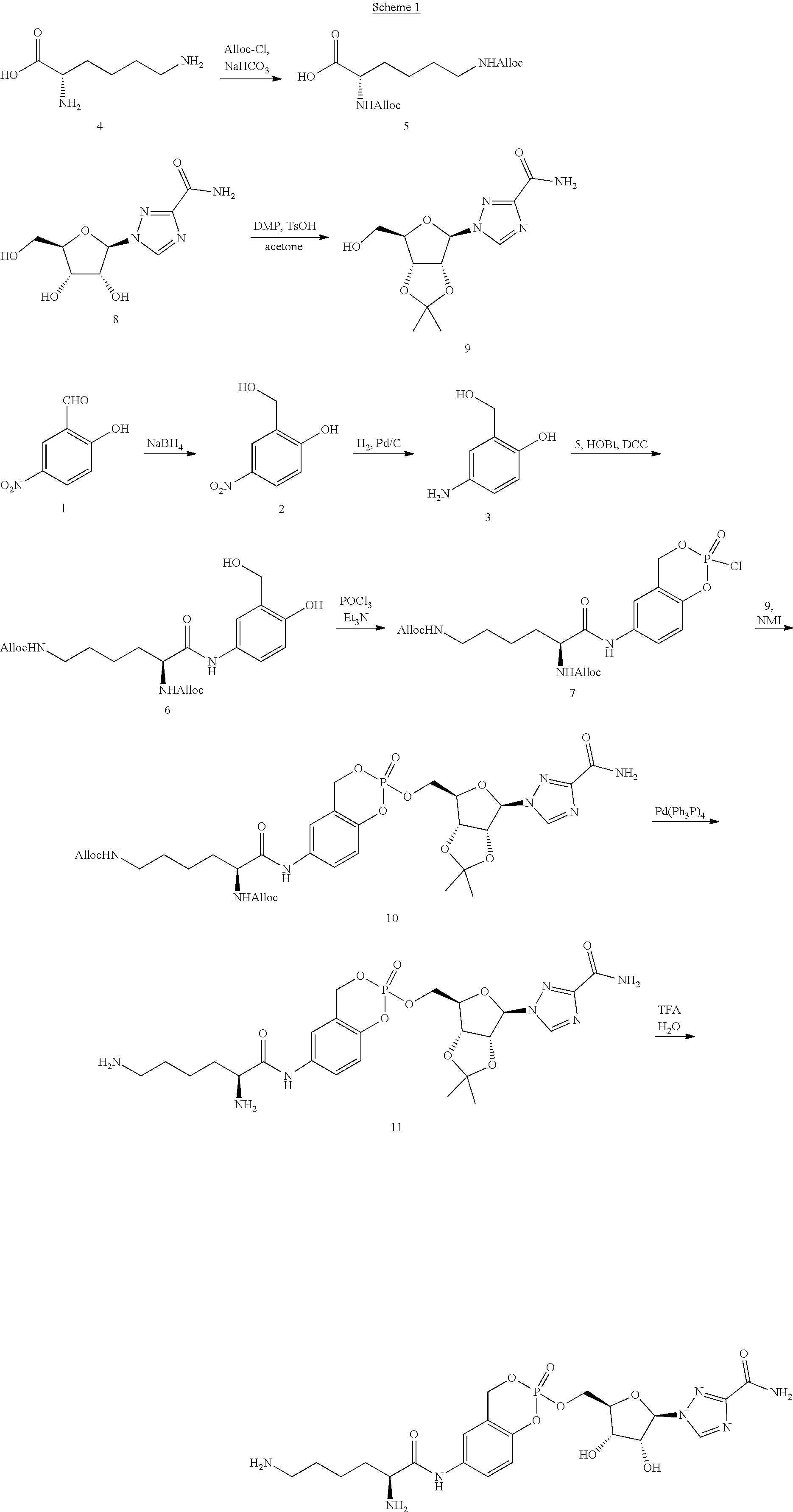

### Step 1

### (2R)-2,6-bis(allyloxycarbonylamino)hexanoic Acid

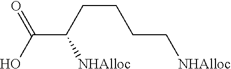

(2R)-2,6-diaminohexanoic acid (4.00 g, 27.36 mmol, 1 eq) was dissolved in H₂O (20.5 mL). The solution was cooled in an ice-bath and treated with NaOH (3.28 g, 82.08 mmol, 3 eq) and allyl carbonochloridate (6.60 g, 54.72 mmol, 5.79 mL, 2 eq). The mixture was stirred at 20° C. for 12 hr. The mixture was acidified to congo red with conc. hydrochloric acid, then extracted with ethyl acetate (3 times, 200 mL each), the organic layer was washed with saturated brine, dried over anhydrous magnesium sulfate, filtered, and the filtrate was concentrated. The residue was purified by column chromatography (SiO2, Dichloromethane:Methanol=100/1 to 20:1) to give (2R)-2,6-bis(allyloxycarbonylamino) hexanoic acid (7.2 g, 22.91 mmol, 83.72% yield) as a colorless oil. (MS: [M+H]⁺ 315.1)

### Step 2

### 2-(hydroxymethyl)-4-nitro-phenol

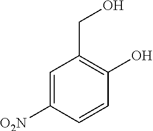 [SMILES: O=[N+]([O-])c1ccc(O)c(CO)c1]

To a solution of 2-hydroxy-5-nitro-benzaldehyde (10 g, 59.84 mmol, 1 eq) in EtOH (100 mL) was added NaBH₄ (4.53 g, 119.68 mmol, 2 eq) in portions. The mixture was stirred at 20° C. for 24 hr. To the reaction mixture was added 200 mL of 3M aq. HCl, and then was extracted with EA (400 mL). The organic layer was concentrated. The residue was purified by column chromatography (SiO2, DCM:MeOH=20:1) to give 2-(hydroxymethyl)-4-nitro-phenol (7 g, 41.39 mmol, 69.17% yield) as yellow solid.

### Step 3

### 4-amino-2-(hydroxymethyl)phenol

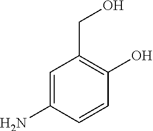

To a solution of 2-(hydroxymethyl)-4-nitro-phenol (4 g, 23.65 mmol, 1 eq) in MeOH (30 mL) was added Pd/C (0.4 g, 2.36 mmol, wt. 10%, 0.1 eq) under N₂ atmosphere. The suspension was degassed and purged with H₂ for 5 times. The mixture was stirred under H₂ (15 Psi) at 20° C. for 12 hr. The mixture was filtered off and the solvent was removed, 4-amino-2-(hydroxy-methyl)phenol (3.5 g, crude) was obtained as slightly purple solid.

### Step 4

### allyl N-[(5R)-5-(allyloxycarbonylamino)-6-[4-hydroxy-3-(hydroxymethyl)anilino]-6-oxo-hexyl]carbamate

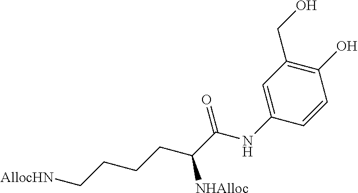

(2R)-2,6-bis(allyloxycarbonylamino)hexanoic acid (7.91 g, 25.15 mmol, 1 eq), 4-amino-2-(hydroxymethyl)phenol (3.5 g, 25.15 mmol, 1 eq) and HOBt (3.74 g, 27.67 mmol, 1.1 eq) were dissolved in dry DMF (10 mL), and then the mixture was cooled to 0° C., DCC (5.71 g, 27.67 mmol, 1.1 eq) was added and the mixture was stirred at 20° C. for 12 hr. The mixture was extracted with ethyl acetate (500 mL*3), the organic layer was washed with saturated brine, dried over anhydrous magnesium sulfate, filtered, and the filtrate was concentrated. The residue was purified by column chromatography (SiO2, Ethyl acetate:Petroleum ether=100/1, 1/1) to give desired allyl N-[(5R)-5-(allyloxycarbonylamino)-6-[4-hydroxy-3-(hydroxy-methyl)anilino]-6-oxo-hexyl]carbamate (5.0 g, 11.48 mmol, 45.65% yield) as slightly purple oil. (MS: [M+H]⁺ 436.1)

### Step 5

### allyl N-[(5S)-5-(allyloxycarbonylamino)-6-[(2-chloro-2-oxo-4H-1,3,2benzodioxa-phosphinin-6-yl)amino]-6-oxo-hexyl]carbamate

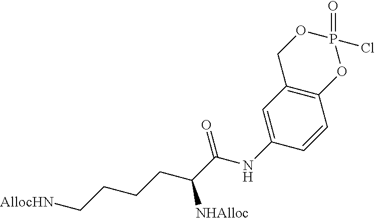

To a solution of allyl N-[(5S)-5-(allyloxycarbonylamino)-6-[4-hydroxy-3-(hydroxymethyl)-anilino]-6-oxo-hexyl]carbamate (2 g, 4.59 mmol, 1 eq) in THF (30 mL) was added TEA (1.39 g, 13.78 mmol, 1.92 mL, 3 eq) in one portion, then POCl₃ (1.06 g, 6.89 mmol, 640.20 μL, 1.5 eq) was added dropwise at −40° C. and stirred at−40˜20° C. for 6 hrs. The reaction mixture was filtered and the solvent was removed under reduced pressure. The residue was used to next step without further purification. allyl N-[(5S)-5-(allyloxycarbonylamino)-6-[(2-chloro-2-oxo-4H-1,3,2benzodioxaphosphinin-6-yl)amino]-6-oxo-hexyl]carbamate (2.2 g, crude) was obtained as yellow oil.

### Step 6

### 1-[(3aR,4R,6R,6aR)-6-(hydroxymethyl)-2,2-dimethyl-3a,4,6,6a-tetrahydrofuro[3,4-d]-[1,3]dioxol-4-yl]-1,2,4-triazole-3-carboxamide

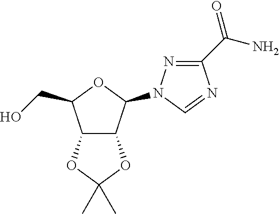 [SMILES: CC1(C)O[C@@H]2[C@H](O1)[C@@H](CO)O[C@H]2n1cnc(C(N)=O)n1]

To a solution of 1-[(2R,3R,4S,5R)-3,4-dihydroxy-5-(hydroxymethyl)tetra-hydrofuran-2-yl]-1,2,4-triazole-3-carboxamide (21 g, 85.99 mmol, 1 eq) in acetone (200 mL) was added 2,2-dimethoxypropane (17.00 g, 163.23 mmol, 20 mL, 1.90 eq) and TsOH·H₂O (2.8 g, 14.72 mmol, 0.17 eq) in one portion, then the mixture was stirred at 70° C. for 0.5 hr. The mixture was diluted with saturated NaHCO₃aq. (15 mL), then it was evaporated to give a residue, which was purified by column chromatography (SiO₂, DCM/MeOH=50/1 to 15:1) to give 1-[(3aR,4R,6R,6aR)-6-(hydroxymethyl)-2,2-dimethyl-3a,4,6,6a-tetrahydrofuro[3,4-d][1,3]-dioxol-4-yl]-1,2,4-triazole-3-carboxamide (21 g, 73.87 mmol, 85.91% yield) as white solid. (MS: [M+H]⁺ 285.1)

### Step 7

### allyl N-[(5S)-6-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzo-dioxaphosphinin-6-yl]amino]-5-(allyloxycarbonylamino)-6-oxo-hexyl]carbamate

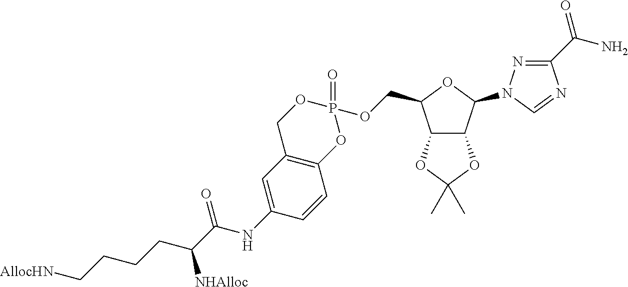

To a solution of 1-[(3aR,4R,6R,6aR)-6-(hydroxymethyl)-2,2-dimethyl-3a,4,6,6a-tetrahydro-furo[3,4-d][1,3]dioxol-4-yl]-1,2,4-triazole-3-carboxamide (1.43 g, 5.04 mmol, 1.3 eq) in DCM (50 mL) was added allyl N-[(5S)-5-(allyloxycarbonylamino)-6-[(2-chloro-2-oxo-4H-1,3,2-benzodioxaphosphinin-6-yl)amino]-6-oxo-hexyl]carbamate (2 g, 3.88 mmol, 1 eq) in one portion, then N-methylimidazole (1.59 g, 19.38 mmol, 5 eq) was added dropwise at 20° C. and was stirred at 20° C. for 1 hr. The reaction mixture was evaporated to give the residue. The residue was purified by reversed-phase column (0.10% HCOOH in 0%˜70% water/CH₃CN) to give the desired peak, after lyophilization, got the crude product. allyl N-[(5S)-6-[[2-[[(3aR,4R,6R,6aR)-4-(3-carbamoyl-1,2,4-triazol-1-yl)-2,2-dimethyl-3a,4,6,6a-tetrahydro-furo[3,4-d][1,3]dioxol-6-yl]methoxy]-2-oxo-4H-1,3,2benzodioxaphosphinin-6-yl]amino]-5-(allyloxycarbonylamino)-6-oxo-hexyl]carbamate (1.5 g, crude) was obtained as yellow solid. (MS: [M+H]⁺ 764.4)

### Step 8

### 1-[(3aR,4R,6R,6aR)-6-[[6-[[(2S)-2,6-diaminohexanoyl]amino]-2-oxo-4H-1,3,2benzo-dioxaphosphinin-2-yl]oxymethyl]-2,2-dimethyl-3a,4,6,6a-tetrahydrofuro[3,4-d][1,3]-dioxol-4-yl]-1,2,4-triazole-3-carboxamide

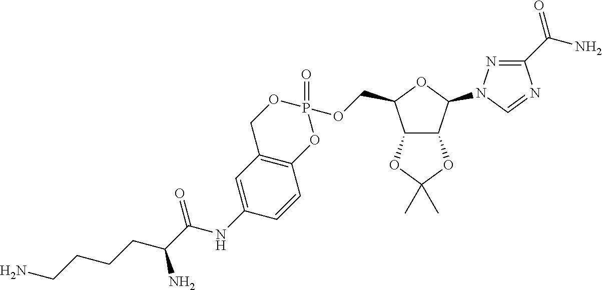 [SMILES: CC1(C)O[C@@H]2[C@H](O1)[C@@H](COP1(=O)OCc3cc(NC(=O)[C@@H](N)CCCCN)ccc3O1)O[C@H]2n1cnc(C(N)=O)n1]

To a solution of allyl N-[(5S)-6-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzodioxaphosphinin-6-yl]amino]-5-(allyloxycarbonylamino)-6-oxo-hexyl]carbamate (500 mg, 654.72 μmol, 1 eq) in DCM (20 mL) was added 1,3-dimethylhexahydropyrimidine-2,4,6-trione (408.91 mg, 2.62 mmol, 4 eq) and Pd(PPh₃)₄ (113.48 mg, 98.21 μmol, 0.15 eq) in one portion. The mixture was stirred at 20° C. for 1 hr. The reaction mixture was evaporated to give the residue. The residue was purified by reversed-phase column (0.1% HCOOH in 0%˜70% water/CH₃CN) to give the desired peak, after lyophilization, got crude 1-[(3aR,4R,6R,6aR)-6-[[6-[[(2S)-2,6-diaminohexanoyl]amino]-2-oxo-4H-1,3,2benzodioxa-phosphinin-2-yl]oxymethyl]-2,2-dimethyl-3a,4,6,6a-tetrahydrofuro[3,4-d][1,3]dioxol-4-yl]-1,2,4-triazole-3-carboxamide (200 mg, crude) as white solid. (MS: [M+H]⁺ 596.3)

### Step 9

### 1-[(2R,3R,4S,5R)-5-[[6-[[(2S)-2,6-diaminohexanoyl]amino]-2-oxo-4H-1,3,2benzodioxa-phosphinin-2-yl]oxymethyl]-3,4-dihydroxy-tetrahydrofuran-2-yl]-1,2,4-triazole-3-carboxamide

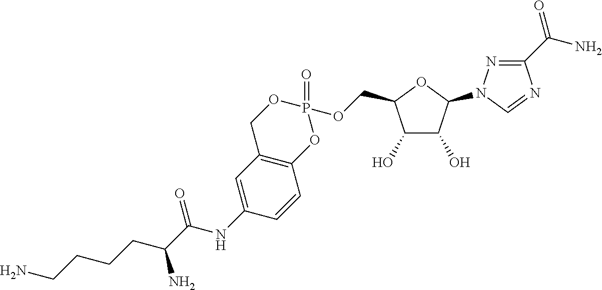 [SMILES: NCCCC[C@H](N)C(=O)Nc1ccc2c(c1)COP(=O)(OC[C@H]1O[C@@H](n3cnc(C(N)=O)n3)[C@H](O)[C@@H]1O)O2]

To a solution of 1-[(3aR,4R,6R,6aR)-6-[[6-[[(2S)-2,6-diaminohexanoyl]amino]-2-oxo-4H-1,3,2benzodioxaphosphinin-2-yl]oxymethyl]-2,2-dimethyl-3a,4,6,6a-tetrahydrofuro[3,4-d][1,3]dioxol-4-yl]-1,2,4-triazole-3-carboxamide (150 mg, 251.87 μmol, 1 eq) in H₂O (0.5 mL) was added TFA (2 mL) in one portion, then the mixture was stirred at 20° C. for 5 min. Water (5 mL) was added into the reaction mixture. The mixture was purified by reversed-phase column (0.1% HCOOH in 0%˜70% water/CH₃CN) to give the desired peak, after lyophilization, got 1-[(2R,3R,4S,5R)-5-[[6-[[(2S)-2,6-diaminohexanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide (22.5 mg, 40.51 μmol, 16.08% yield) as white solid. (MS: [M+H]⁺ 556.3); ¹H NMR (MeOD, 400 MHz): δ (ppm) 8.58-8.54 (m, 1H), 8.34 (s, 1H), 7.50-7.41 (m, 2H), 7.02-6.99 (m, 1H), 5.92-5.90 (m, 1H), 5.45-5.31 (m, 3H), 4.51-4.42 (m, 5H), 3.95-3.94 (m, 1H), 2.99-2.97 (m, 3H), 2.05-1.95 (m, 2H), 1.76-1.73 (m, 3H), 1.59-1.56 (m, 3H); ³¹P NMR (MeOD, 162 MHz): δ (ppm) −9.43, −9.63.

### Example 5

### Synthesis of 1-[(2R,3R,4S,5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide Using Scheme 2

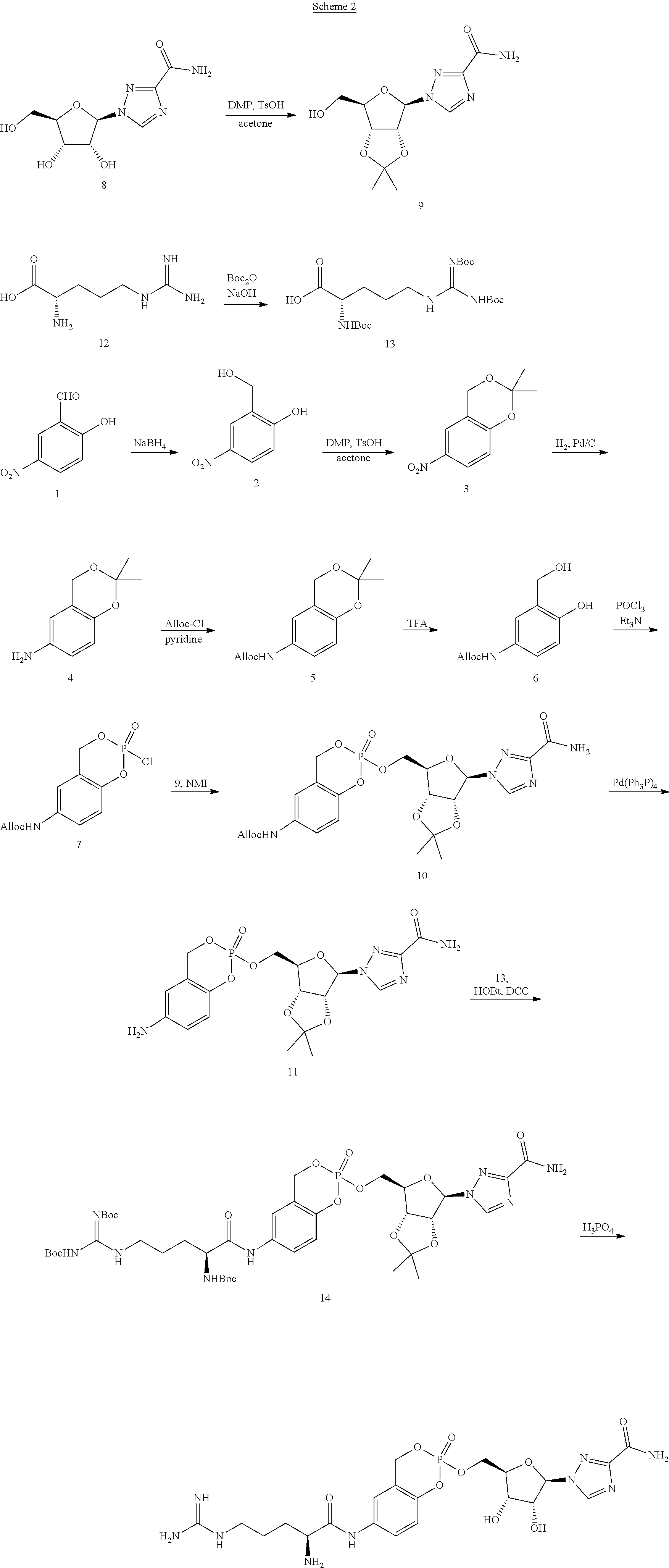

### Step 1

### (2S)-5-[[(Z)-N,N′-bis(tert-butoxycarbonyl)carbamimidoyl]amino]-2-(tert-butoxy-carbonylamino)pentanoic Acid

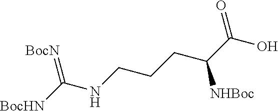

(2S)-2-amino-5-guanidino-pentanoic acid (17.4 g, 99.88 mmol, 1 eq) was dissolved in t-BuOH (250 mL) and H2O (250 mL), then NaOH (14 g, 350.03 mmol, 3.50 eq) was added in portions at 0° C., then Boc2O (87.20 g, 399.54 mmol, 91.79 mL, 4 eq) was added in portions at 0° C., the mixture was stirred at 25° C. for 24 hr, The reaction mixture was evaporated to about ½ volume, then EtOAc (200 mL) was added, EtOAc phase was discarded, then carefully acidified with citric acid to pH 3 and was extracted with ethyl acetate (100 mL×2). The extract was dried with anhydrous sodium sulfate and evaporated to give the residue. The residue was dried in a vacuum oven to give (2S)-5-[[(Z)-N,N′-bis(tert-butoxycarbonyl)carbamimidoyl]amino]-2-(tert-butoxycarbonylamino)pentanoic acid (20 g, 42.15 mmol, 42.19% yield) as white solid.

### Step 2

### 2,2-dimethyl-6-nitro-4H-1,3-benzodioxine

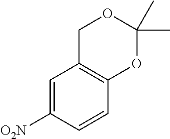

To a solution of 2-(hydroxymethyl)-4-nitro-phenol (9 g, 53.21 mmol, 1 eq) in Acetone (300 mL) was added TsOH·H₂O (2.02 g, 10.64 mmol, 0.2 eq) and 2,2-dimethoxypropane (11.64 g, 111.75 mmol, 13.69 mL, 2.1 eq) in one portion. Then the mixture was stirred at 40° C. for 8 hr. The mixture was quenched with NH₃·H₂O (3 mL), and then was evaporated to give the residue. The residue was purified by column chromatography (SiO2, PE/EtOAc=10/1 to 5:1) to obtain 2,2-dimethyl-6-nitro-4H-1,3-benzodioxine (9 g, 43.02 mmol, 80.85% yield) as yellow solid.

### Step 3

### 2,2-dimethyl-4H-1,3-benzodioxin-6-amine

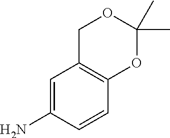

2,2-dimethyl-6-nitro-4H-1,3-benzodioxine (9 g, 43.02 mmol, 1 eq) was dissolved in MeOH (150 mL) and then Pd/C (1 g, 10% purity) was added in one portion, the mixture was stirred at 20° C. under H2 at 15 psi for 2 hr. The reaction mixture was filtered to give the filtrate. The filtrate was evaporated to give the crude 2,2-dimethyl-4H-1,3-benzodioxin-6-amine (7.6 g, crude) as yellow solid.

### Step 4

### allyl N-(2,2-dimethyl-4H-1,3-benzodioxin-6-yl)carbamate

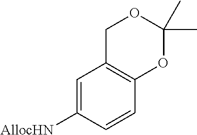

2,2-dimethyl-4H-1,3-benzodioxin-6-amine (7.5 g, 41.85 mmol, 1 eq) was dissolved in DCM (200 mL) and then pyridine (9.93 g, 125.55 mmol, 10.13 mL, 3 eq) was added in one portion, then allyl carbonochloridate (7.57 g, 62.77 mmol, 6.64 mL, 1.5 eq) was added in portions at 0° C. and stirred at 20° C. for 2 hr. The reaction mixture was quenched with saturated aq. NaHCO₃ (100 mL), then extracted with EtOAc (200 mL*2) and the solvent was evaporated to give the residue. The residue was purified by column chromatography (SiO2, PE/EA=8/1 to 5:1) to obtain allyl N-(2,2-dimethyl-4H-1,3-benzodioxin-6-yl)carbamate (10.5 g, 39.88 mmol, 95.30% yield) as yellow oil.

### Step 5

### allyl N-[4-hydroxy-3-(hydroxymethyl)phenyl]carbamate

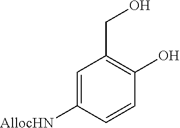

Allyl N-(2,2-dimethyl-4H-1,3-benzodioxin-6-yl)carbamate (9 g, 34.18 mmol, 1 eq) was dissolved in AcOH (60 mL) and H₂O (30 mL), then the mixture was stirred at 70° C. for 0.5 hr. The reaction mixture was quenched with saturated NaHCO₃ (400 mL), then was extracted with EtOAc (300 mL*2), the organic solvent was washed with water (200 mL) and then was washed with brine (200 mL), then the solvent was evaporated to give the residue. The residue was used to next step without further purification. allyl N-[4-hydroxy-3-(hydroxymethyl)-phenyl]carbamate (7.5 g, 33.60 mmol, 98.29% yield) was obtained as white solid.

### Step 6

### allyl N-(2-chloro-2-oxo-4H-1,3,2benzodioxaphosphinin-6-yl)carbamate

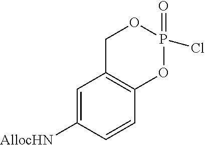

To a solution of allyl N-[4-hydroxy-3-(hydroxymethyl)phenyl]carbamate (3 g, 13.44 mmol, 1 eq) in THF (80 mL) was added TEA (4.08 g, 40.32 mmol, 5.61 mL, 3 eq) in one portion. Then POCl₃ (3.09 g, 20.16 mmol, 1.87 mL, 1.5 eq) was added dropwise at −40° C. and stirred at −40˜20° C. for 6 hr. The reaction mixture was filtered and the solvent was removed under reduced pressure using a high-vacuum pump. The residue was used to next step without further purification. allyl N-(2-chloro-2-oxo-4H-1,3,2benzodioxaphosphinin-6-yl)carbamate (3.5 g, crude) was obtained as yellow oil. (MS: [M+H]⁺ 304.0)

### Step 7

### allyl N-[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]carbamate

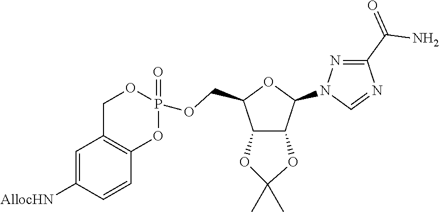

To a solution of 1-[(3aR,4R,6R,6aR)-6-(hydroxymethyl)-2,2-dimethyl-3a,4,6,6a-tetrahydro-furo[3,4-d][1,3]dioxol-4-yl]-1,2,4-triazole-3-carboxamide (3.5 g, 12.31 mmol, 1.10 eq.) in DCM (100 mL) was added allyl N-(2-chloro-2-oxo-4H-1,3,2benzodioxaphosphinin-6-yl)carbamate (3.4 g, 11.20 mmol, 1 eq) in one portion. Then N-methylimidazole (2.76 g, 33.59 mmol, 2.68 mL, 3 eq) was added dropwise at 20° C. and stirred at 20° C. for 1 hr, the reaction mixture was evaporated to give the residue. The residue was purified by reversed-phase column (0.1% HCOOH in 0%˜70% water/CH₃CN) to give the desired peak, after lyophilization, got allyl N-[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzodioxaphosphinin-6-yl]carbamate (3.0 g, 5.44 mmol, 48.58% yield) as yellow solid. (MS: [M+H]⁺ 552.3)

### Step 8

### 1-[(3aR,4R,6R,6aR)-6-[(6-amino-2-oxo-4H-1,3,2benzodioxaphosphinin-2-yl)oxymethyl]-2,2-dimethyl-3a,4,6,6a-tetrahydrofuro[3,4-d][1,3]dioxol-4-yl]-1,2,4-triazole-3-carboxamide

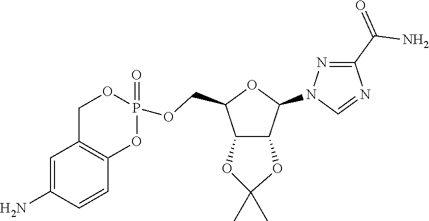 [SMILES: CC1(C)O[C@@H]2[C@H](O1)[C@@H](COP1(=O)OCc3cc(N)ccc3O1)O[C@H]2n1cnc(C(N)=O)n1]

To a solution of allyl N-[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo4H-1,3,2-benzo-dioxaphosphinin-6-yl]carbamate (800 mg, 1.45 mmol, 1 eq) in DCM (100 mL) was added 1,3-dimethylhexahydropyrimidine-2,4,6-trione (906.08 mg, 5.80 mmol, 4 eq) and Pd(PPh₃)₄ (251.46 mg, 217.61 μmol, 0.15 eq) in one portion. Then the mixture was stirred at 20° C. for 1 hr. The reaction mixture was evaporated to give the residue. The residue was used to next step without further purification. 1-[(3aR,4R,6R,6aR)-6-[(6-amino-2-oxo-4H-1,3,2benzodioxa-phosphinin-2-yl)oxymethyl]-2,2-dimethyl-3a,4,6,6a-tetrahydrofuro[3,4-d][1,3]dioxol-4-yl]-1,2,4-triazole-3-carboxamide (660 mg, crude) was obtained as yellow solid. (MS: [M+H]⁺ 468.3).

### Step 9

### tert-butyl (NZ)-N-[[[(4S)-5-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzodioxaphosphinin-6-yl]amino]-4-(tert-butoxycarbonylamino)-5-oxo-pentyl]amino]-(tert-butoxycarbonylamino)methylene]carbamate

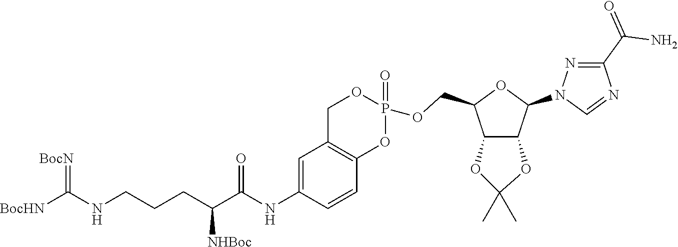

To a solution of 1-[(3aR,4R,6R,6aR)-6-[(6-amino-2-oxo-4H-1,3,2benzodioxaphosphinin-2-yl)oxymethyl]-2,2-dimethyl-3a,4,6,6a-tetrahydrofuro[3,4-d][1,3]dioxol-4-yl]-1,2,4-triazole-3-carboxamide (660 mg, 1.41 mmol, 1 eq) in THF (50 mL) was added (2S)-5-[[(Z)-N,N′-bis-(tert-butoxycarbonyl)carbamimidoyl]amino]-2-(tert-butoxycarbonylamino)pentanoic acid (871.18 mg, 1.84 mmol, 1.3 eq) and HOBt (286.22 mg, 2.12 mmol, 1.5 eq) in one portion, then DCC (437.05 mg, 2.12 mmol, 1.5 eq) was added in one portion at 0° C. Then the mixture was stirred at 20° C. for 4 hr. The reaction mixture was evaporated to give the residue. The residue was purified by reversed-phase column (0.1% NH3·H2O in 0%˜70% water/CH₃CN) to give the desired peak, after lyophilization, got desired product. tert-butyl (NZ)-N-[[[(4S)-5-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]amino]-4-(tert-butoxycarbonylamino)-5-oxo-pentyl]amino]-(tert-butoxycarbonylamino)-methylene]carbamate (550 mg, 595.30 μmol, 42.16% yield) as yellow solid. (MS: [M+H]⁺ 924.4)

### Step 10

### 1-[(2R,3R,4S,5R)-5-[[6-[[(2S)-2-amino-5-guanidino-pentanoyl]amino]-2-oxo-4H-1,3,2-benzodioxaphosphinin-2-yl]oxymethyl]-3,4-dihydroxy-tetrahydrofuran-2-yl]-1,2,4-triazole-3-carboxamide

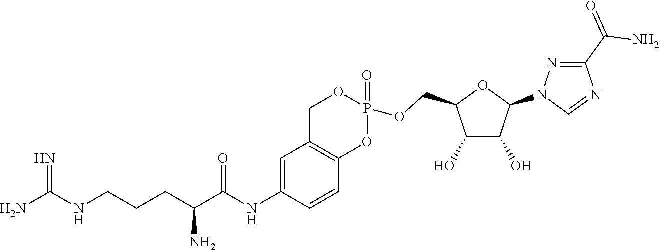 [SMILES: N=C(N)NCCC[C@H](N)C(=O)Nc1ccc2c(c1)COP(=O)(OC[C@H]1O[C@@H](n3cnc(C(N)=O)n3)[C@H](O)[C@@H]1O)O2]

Tert-butyl (NZ)-N-[[[(4S)-5-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzo-dioxaphosphinin-6-yl]amino]-4-(tert-butoxycarbonylamino)-5-oxo-pentyl]amino]-(tert-butoxycarbonylamino)methylene]carbamate (100 mg, 108.24 μmol, 1 eq) was dissolved in H₃PO₄ (1.68 g, 14.57 mmol, 1.00 mL, 85% purity, 134.63 eq), then the mixture was stirred at 20° C. for 0.5 hr. Water (4 mL) was added to the mixture. The mixture was purified by reversed-phase column (0.1% HCOOH in 0%˜70% water/CH₃CN) to give the desired peak, after lyophilization, got 1-[(2R,3R,4S,5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide (16.7 mg, 26.04 μmol, 24.06% yield, 91% purity) as white solid. (MS: [M+H]⁺ 584.3); ¹H NMR (MeOD, 400 MHz): δ (ppm) 8.55 (s, 1H), 8.51 (s, 1H), 8.39 (s, 1H), 7.31-7.25 (m, 1H), 7.18-7.17 (m, 1H), 7.00-6.89 (m, 1H), 5.98 (s, 1H), 5.39-5.31 (m, 2H), 4.69-4.63 (m, 1H), 4.53-4.29 (m, 4H), 4.10-4.09 (m, 1H), 3.23-3.20 (m, 2H), 2.01-1.96 (m, 2H), 1.71-1.65 (m, 2H); ³¹P NMR (MeOD, 162 MHz): δ (ppm) −7.60, −8.07.

### Example 6

### Synthesis of 4-[[(1S)-2-[[(1S)-2-[[(1S)-1-benzyl-2-[[2-[[(2R,3S,4R,5R)-5-(3-carbamoyl-1,2,4-triazol-1-yl)-3,4-dihydroxy-tetrahydrofuran-2-yl]methoxyl-2-oxo-4H-1,3,2benzodioxaphosphinin-6-yl]amino]-2-oxo-ethyl]amino]-1-methyl-2-oxo-ethyl]amino]-1-methyl-2-oxo-ethyl]amino]-4-oxo-butanoic acid Using Scheme 3

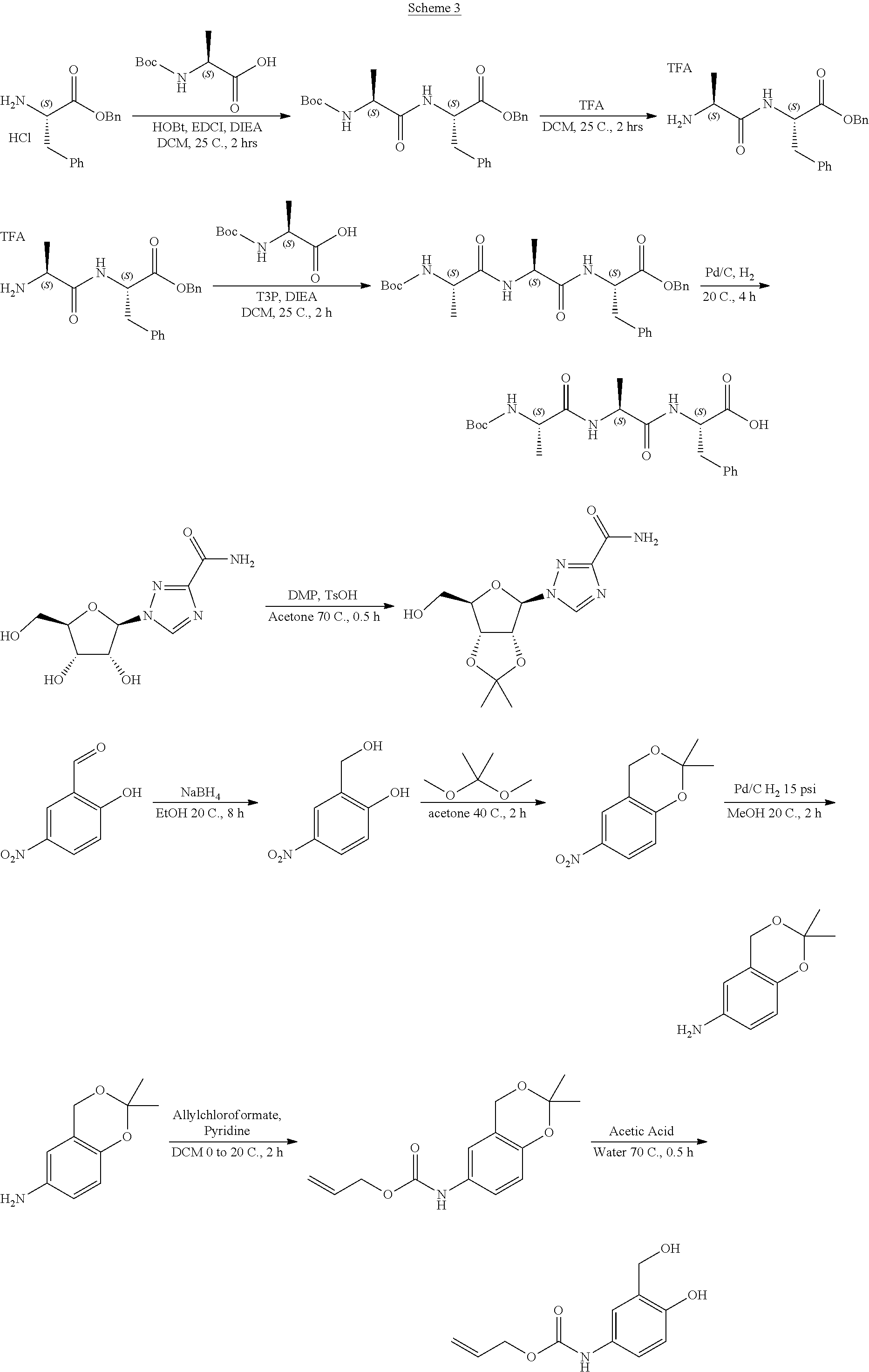

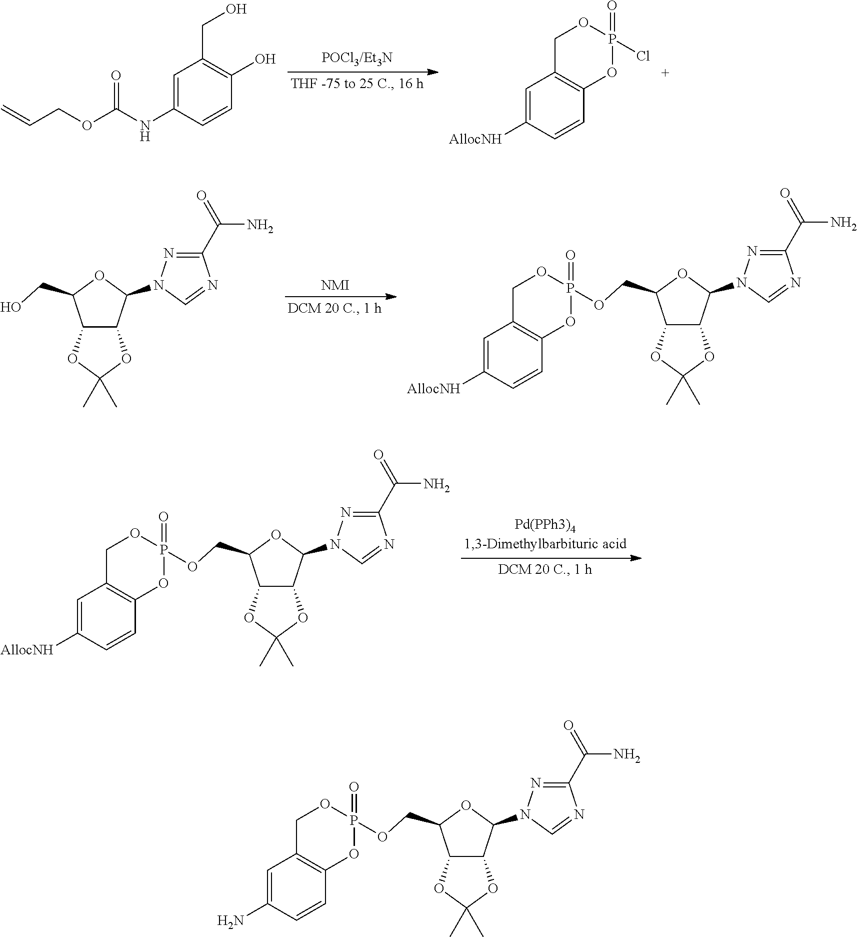

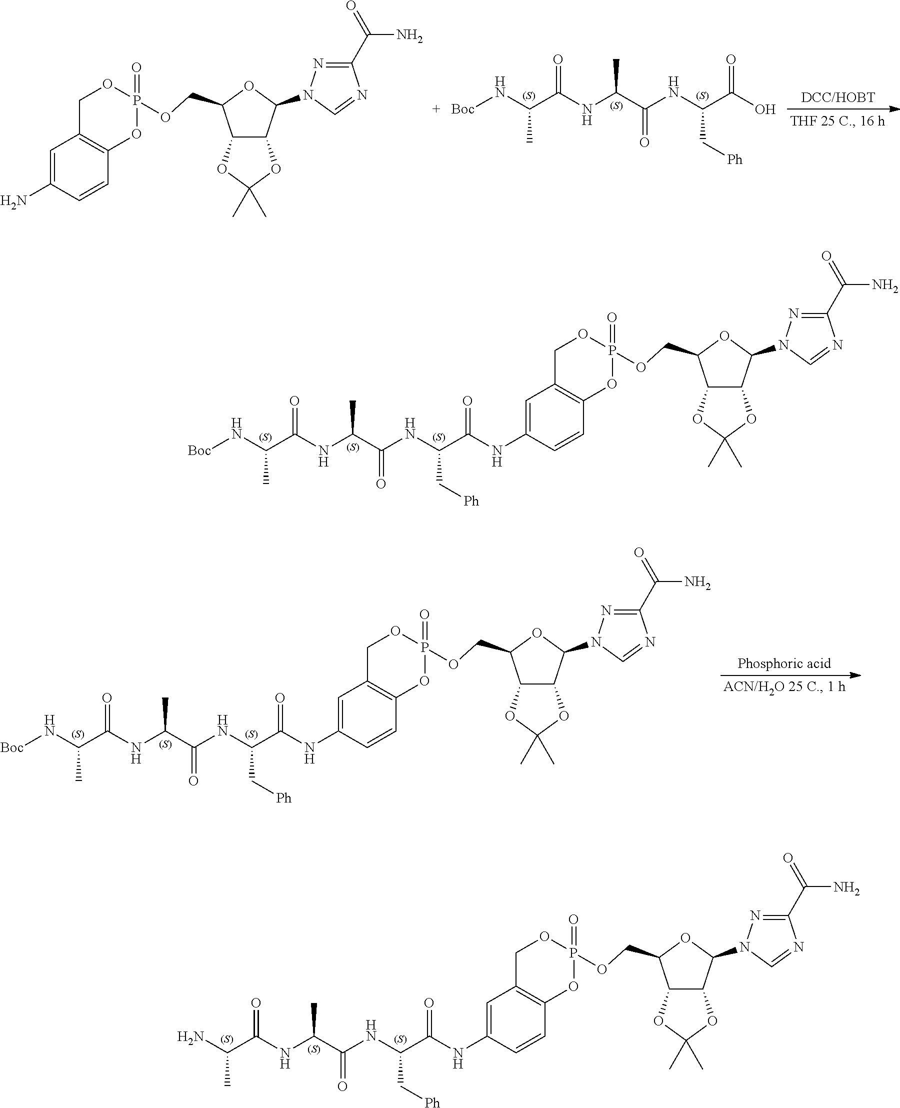

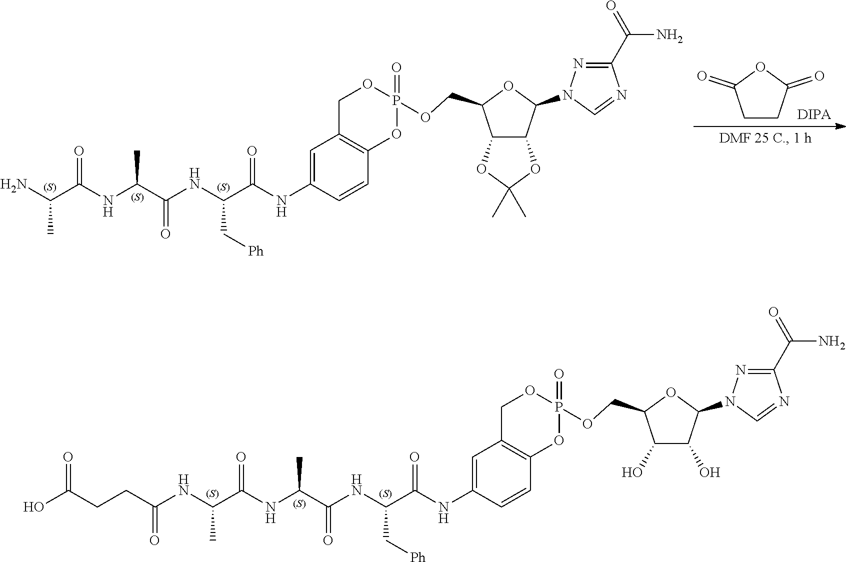

### Step 1

### Benzyl (2S)-2-[(2S)-2-(tert-butoxycarbonylamino)propionylamino]-3-phenylpropionate

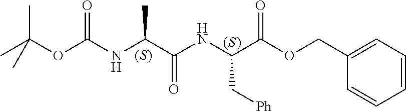

A solution of (S)-2-(tert-Butoxycarbonylamino)propionic acid (3.78 g, 20 mmol), EDCI (4.601 g, 24 mmol), and HOBt (3.243 g, 24 mmol) in 150 mL of DCM was cooled to 0 C. To this solution was added DIEA (9.305 g, 72 mmol), and then Benzyl (S)-2-amino-3-phenylpropionate (5.1 g, 20 mmol). The solution was brought up to 25C and stirred for 2 h. Then 100 mL of water was added to the reaction mixture and separated. The aqueous layer was further extracted with DCM (150 mL×3). The organic layers were combined and washed with 1.0 N NaOH, 1.0 N HCl, and brine subsequently, and then dried over Na₂SO₄ before being concentrated to dryness in vacuo. The crude product was purified by flash chromatography to give Benzyl (2S)-2-[(2S)-2-(tert-butoxycarbonylamino)propionylamino]-3-phenylpropionate.

### Step 2

### Benzyl (2S)-2-[(2S)-2-aminopropionylamino]-3-phenylpropionate trifluoroacetate Salt

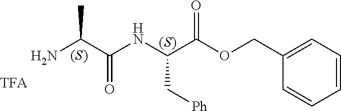

Benzyl (2S)-2-[(2S)-2-(tert-butoxycarbonylamino)propionylamino]-3-phenylpropionate (4.1 g, 9.5 mmol) was dissolved in DCM (7 mL). TFA (3.5 mL) was added to the solution and stirred for 2 h at room temperature. The solution was evaporated under vacuum to remove DCM and TFA. The resulting residue was mostly redissolved in acetonitrile and this suspension was filtered. Diethyl ether was added to the filtrate to precipitate a solid. The solid was filtered again and dried in vacuo yielding Benzyl (2S)-2-[(2S)-2-aminopropionylamino]-3-phenylpropionate trifluoroacetate salt.

### Step 3

### Benzyl (2S)-2-{(2S)-2-[(2S)-2-(tert-butoxycarbonylamino)propionylamino]propionylamino}-

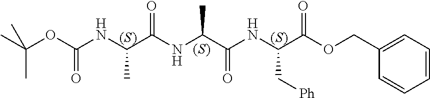

To a solution of (S)-2-(tert-Butoxycarbonylamino)propionic acid (624 mg, 3.3 mmol) and Benzyl (2S)-2-[(2S)-2-aminopropionylamino]-3-phenylpropionate trifluoroacetate salt (1.1 g, 2.5 mmol) cooled to 0 C in DCM (10 mL) was added T3P (2.2 g, 3.3 mmol) and DIPEA (646 mg, 5.0 mmol). The reaction mixture was allowed to warm to 25C and stirred for 2 h. The reaction mixture was extracted by DCM (2×30 mL). The organic phase was washed with 5% aqueous NaHCO₃ (10 mL), aqueous HCl (5 mL, 1 M), and saturated aqueous brine solution (2×30 mL). The organic phase was dried over Na₂SO₄ and concentrated in vacuo. The residue was purified by column chromatography on silica gel to give Benzyl (2S)-2-{(2S)-2-[(2S)-2-(tert-butoxycarbonylamino)propionylamino]propionylamino}-3-henylpropionate.

### Step 4

### (2S)-2-[[(2S)-2-[[(2S)-2-(tert-butoxycarbonylamino)propanoyl]amino]propanoyl]amino]-3-phenyl-propanoic Acid

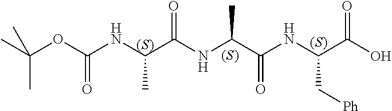

(2S)-2-{(2S)-2-[(2S)-2-(tert-butoxycarbonylamino)propionylamino]propionyl-amino}-3-phenylpropionate (4.2 g, 8.45 mmol) was added to a 100 mL solution of MeOH, followed by Pd/C (10% w/w; 200 mg) and the resulting black suspension was sparged with N₂ (3×) and placed under 1 atm of H₂. The mixture was stirred at room temperature overnight and filtered to remove the catalyst. The resulting clear solution was then concentrated in vacuo to obtain (2S)-2-[[(2S)-2-[[(2S)-2-(tert-butoxycarbonylamino)propanoyl amino]propanoyl]-amino]-3-phenyl-propanoic acid, which was used without further purification.

### Step 5

### Tert-butyl N-[(1S)-2-[[(1S)-2-[[(1S)-2-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]amino]-1-benzyl-2-oxo-ethyl]amino]-1-methyl-2-oxo-ethyl]amino]-1-methyl-2-oxo-ethyl]carbamate

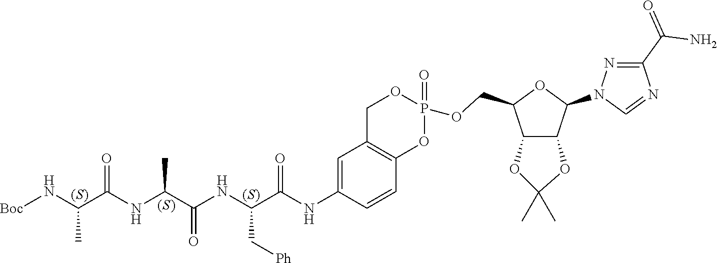

To a solution of 1-[(3aR,4R,6R,6aR)-6-[(6-amino-2-oxo-4H-1,3,2benzodioxaphosphinin-2-yl)oxymethyl]-2,2-dimethyl-3a,4,6,6a-tetrahydrofuro[3,4-d][1,3]dioxol-4-yl]-1,2,4-triazole-3-carboxamide (250 mg, 534.91 umol, 1 eq) [see example 5 vide supera for synthesis] in THF (2 mL) was added DCC (165.55 mg, 802.36 umol, 1.5 eq) and HOBt (108.42 mg, 802.36 umol, 1.5 eq). After stirring for 10 min, (2S)-2-[[(2S)-2-[[(2S)-2-(tert-butoxycarbonylamino)propanoyl]amino]propanoyl]amino]-3-phenyl-propanoic acid (239.75 mg, 588.40 umol, 1.1 eq) was added. The mixture was stirred at 25° C. for 16 hours. The reaction mixture was concentrated in vacuo. The crude product was purified by reverse-phase HPLC (0.1% FA condition, H₂O/ACN, 0˜70%) and lyophilized to yield tert-butyl N-[(1S)-2-[[((1S)-2-[[((1S)-2-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]amino]-1-benzyl-2-oxo-ethyl]amino]-1-methyl-2-oxo-ethyl]amino]-1-methyl-2-oxo-ethyl]carbamate (200 mg, 233.42 umol, 43.64% yield) as a light yellow solid. (MS: [M+H]⁺ 857.4)

### Step 6

### 1-[(2R,3R,4S,5R)-5-[[6-[[(2S)-2-[[(2S)-2-[[(2S)-2-aminopropanoyl]amino]propanoyl]amino]-3-phenyl-propanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide

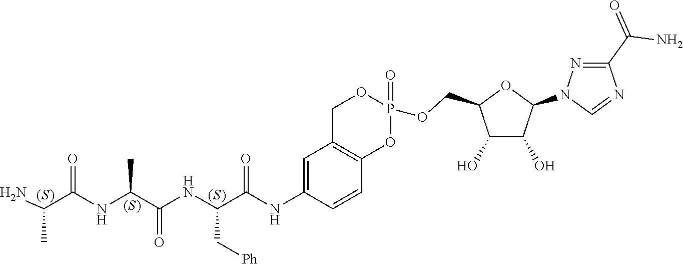

To a solution of tert-butyl N-[(1S)-2-[[(1S)-2-[[(1S)-2-[[2-[[(3aR,4R,6R,6aR)-4-(3-carbamoyl-1,2,4-triazol-ylz0-2,2-dimethyl-3a,4,6,6a-tetrahydrofurol[3,4-d][1,3]dioxol-6-yl]-methoxy]-2-oxo-4H-1,3,2benzodioxaphosphinin-6-yl]amino]-1-benzyl-2-oxo-ethyl]amino]-1-methyl-2-oxo-ethyl]amino]-1-methyl-2-oxo-ethyl]carbamate (100.12 mg, 116.85 umol, 1 eq) in ACN (0.2 mL) and H₂O (1 mL) was added H₃PO₄ (1 mL, 80% purity). The mixture was stirred at 25° C. for 1 hour. The crude product was purified by reversed-phase HPLC (0.1% FA condition, H2O/ACN, 0˜50%) and lyophilized. Compound 1-[(2R,3R,4S,5R)-5-[[6-[[(2S)-2-[[(2S)-2-[[(2S)-2-aminopropanoyl]amino]propanoyl]amino]-3-phenyl-propanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide (55 mg, 71.51 umol, 61.20% yield, 99.156% purity, FA) was obtained as a white solid. (MS: [M+H]⁺ 717.3) ¹H NMR (400 MHz, DMSO-d6) δ=10.24-10.11 (m, 1H), 8.79 (s, 1H), 8.41-8.21 (m, 4H), 7.86 (s, 1H), 7.64 (s, 1H), 7.58-7.41 (m, 2H), 7.32-6.98 (m, 6H), 5.89 (t, J=2.4 Hz, 1H), 5.44 (br d, J=12.2 Hz, 3H), 4.71-4.52 (m, 2H), 4.35-4.22 (m, 6H), 3.08-3.03 (m, 1H), 2.90 (dd, J=9.3, 13.8 Hz, 1H), 1.18-1.12 (m, 7H). ³¹P NMR (400 MHz, DMSO-d6): δ (ppm) −9.790 ppm

### Step 7

### 4-[[(1S)-2-[[(1S)-2-[[(1S)-1-benzyl-2-[[2-[[(2R,3S,4R,5R)-5-(3-carbamoyl-1,2,4-triazol-1-yl)-3,4-dihydroxy-tetrahydrofuran-2-yl]methoxy]-2-oxo-4H-1,3,2benzodioxaphosphinin-6-yl]amino]-2-oxo-ethyl]amino]-1-methyl-2-oxo-ethyl]amino]-1-methyl-2-oxo-ethyl]amino]-4-oxo-butanoic Acid

To a solution of tert-butyl N-[(1S)-2-[[(1S)-2-[[(1S)-2-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]amino]-1-benzyl-2-oxo-ethyl]amino]-1-methyl-2-oxo-ethyl]amino]-1-methyl-2-oxo-ethyl]carbamate (100.12 mg, 116.85 umol, 1 eq) in ACN (0.2 mL) and H₂O (1 mL) was added H₃PO₄ (1 mL, 80% purity)

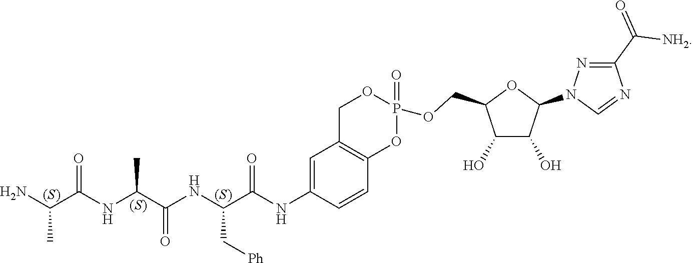

The mixture was stirred at 25° C. for 1 hour. The crude product was purified by reversed-phase HPLC (0.1% FA condition, H2O/ACN, 0˜50%) and lyophilized. Compound 1-[(2R,3R,4S,5R)-5-[[6-[[(2S)-2-[[(2S)-2-[[(2S)-2-aminopropanoyl]amino]propanoyl]amino]-3-phenyl-propanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide (55 mg, 71.51 umol, 61.20% yield, 99.156% purity, FA) was obtained as a white solid. (MS: [M+H]⁺ 717.3)

### Step 8

### 4-[[(1S)-2-[[(1S)-2-[[(1S)-1-benzyl-2-[[2-[[(2R,3S,4R,5R)-5-(3-carbamoyl-1,2,4-triazol-1-yl)-3,4-dihydroxy-tetrahydrofuran-2-yl]methoxy]-2-oxo-4H-1,3,2benzodioxaphosphinin-6-yl]amino]-2-oxo-ethyl]amino]-1-methyl-2-oxo-ethyl]amino]-1-methyl-2-oxo-ethyl]amino]-4-oxo-butanoic Acid

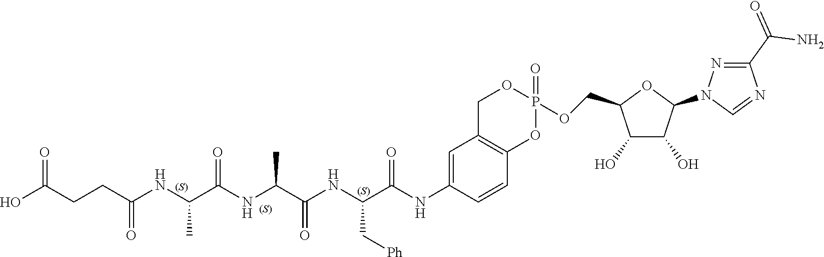

To a solution of 1-[(2R,3R,4S,5R)-5-[[6-[[(2S)-2-[[(2S)-2-[[(2S)-2-aminopropanoyl]amino]propanoyl]amino]-3-phenyl-propanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide (50 mg, 69.77 umol, 1 eq) in DMF (0.3 mL) was added DIPEA (18.03 mg, 139.54 umol, 24.30 uL, 2 eq) and tetrahydrofuran-2,5-dione (6.98 mg, 69.77 umol, 1 eq). The mixture was stirred at 25° C. for 1 hour. The mixture was concentrated in vacuum. The crude product was purified by reversed-phase HPLC (0.1% FA condition, H₂O/ACN, 0-40%) and lyophilized. Compound 4-[[(1S)-2-[[(1S)-2-[[(1S)-1-benzyl-2-[[2-[[(2R,3S,4R,5R)-5-(3-carbamoyl-1,2,4-triazol-1-yl)-3,4-dihydroxy-tetrahydrofuran-2-yl]methoxy]-2-oxo-4H-1,3,2benzodioxaphosphinin-6-yl]amino]-2-oxo-ethyl]amino]-1-methyl-2-oxo-ethyl]amino]-1-methyl-2-oxo-ethyl]amino]-4-oxo-butanoic acid (10.5 mg, 12.74 umol, 18.25% yield, 99.062% purity) was obtained as a white solid. (MS: [M+H]⁺ 817.4).

¹H NMR (400 MHz, DMSO-d6) δ=12.41-11.82 (m, 1H), 9.98 (s, 1H), 8.79 (s, 1H), 8.15 (d, J=6.8 Hz, 1H), 8.06 (br d, J=7.0 Hz, 1H), 7.99 (d, J=7.1 Hz, 1H), 7.86 (s, 1H), 7.63 (s, 1H), 7.54 (dd, J=2.3, 16.1 Hz, 1H), 7.50-7.42 (m, 1H), 7.30-7.15 (m, 4H), 7.11-6.99 (m, 1H), 5.89 (t, J=2.6 Hz, 1H), 5.82-5.61 (m, 1H), 5.51-5.34 (m, 2H), 4.67-4.48 (m, 1H), 4.44-3.97 (m, 7H), 3.08 (dd, J=5.4, 13.8 Hz, 1H), 2.99-2.87 (m, 1H), 2.46-2.31 (m, 4H), 1.16 (dd, J=7.1, 11.7 Hz, 6H)

³¹P NMR (400 MHz, DMSO-d6): δ (ppm) −9.792 ppm

### Example 7

### Synthesis of (S)-2-(p-{[(2-{(S)-2-[(2S,3R,4S,5S,6R)-3,4,5-Trihydroxy-6-(hydroxymethyl)tetrahydro-2H-pyran-2-yloxy]-4-methylvalerylamino}-4-amino-6-pteridinyl)methyl]-N-methylamino}benzylamino)glutaric Acid Using Scheme 4

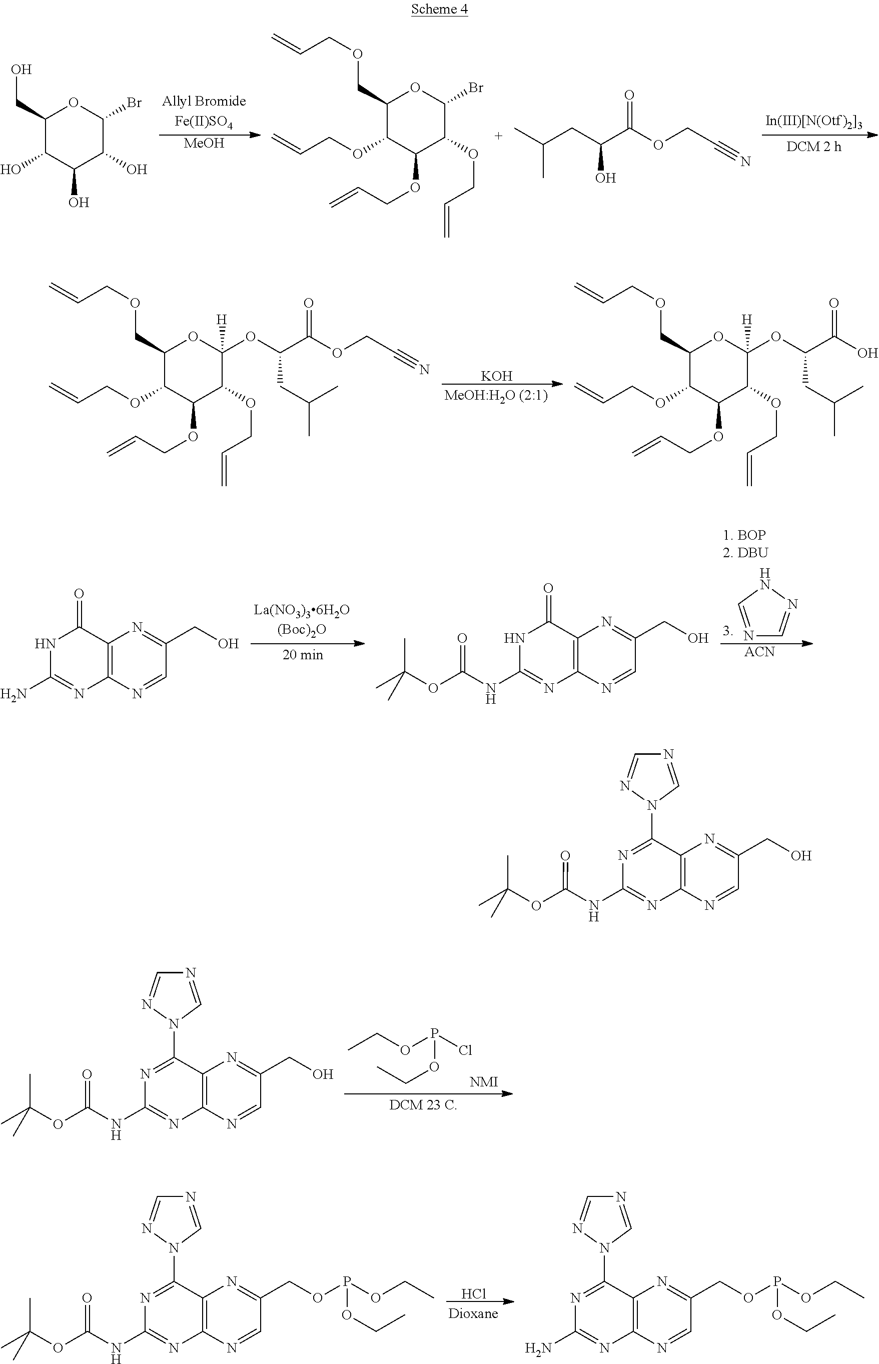

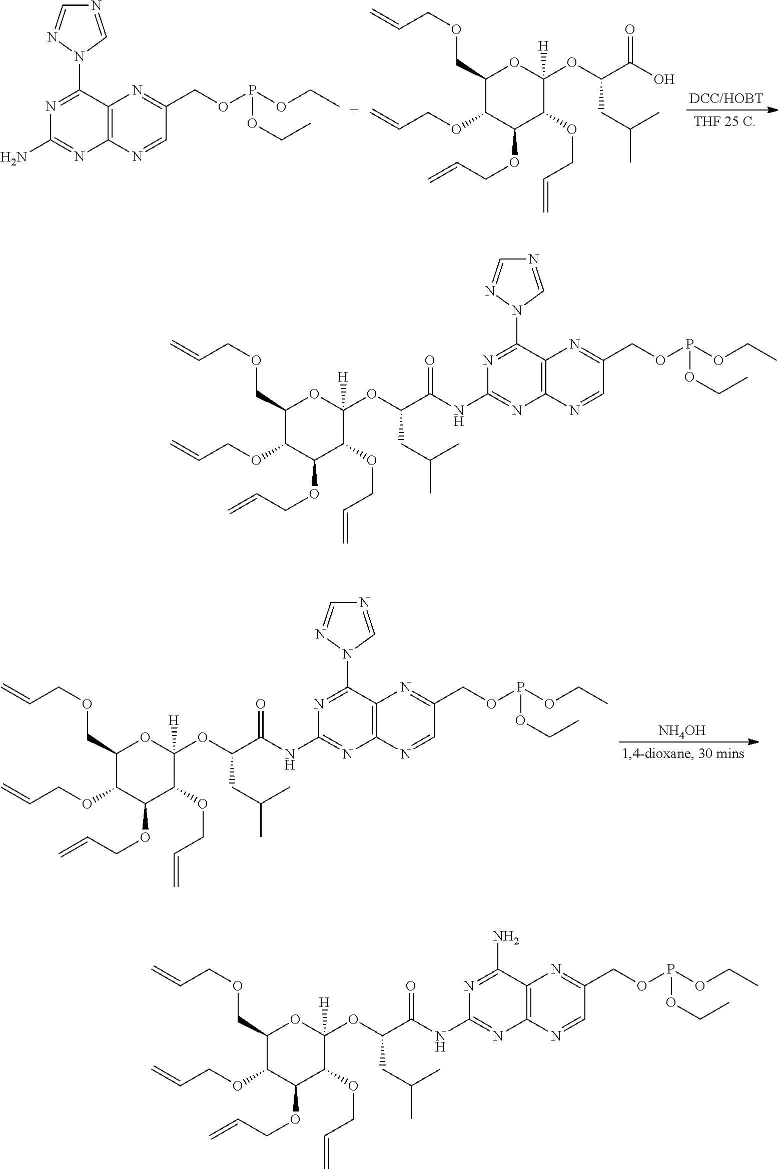

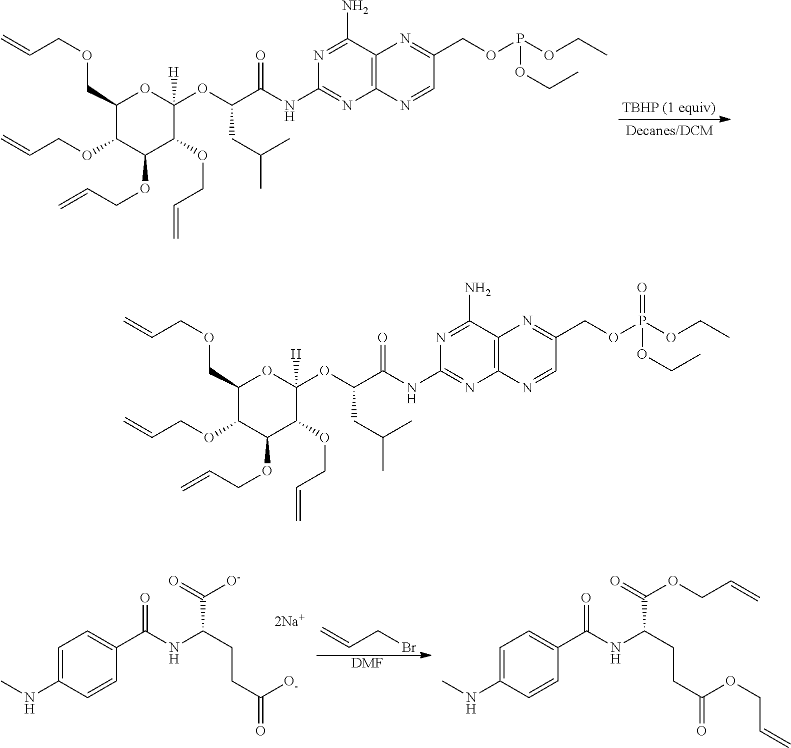

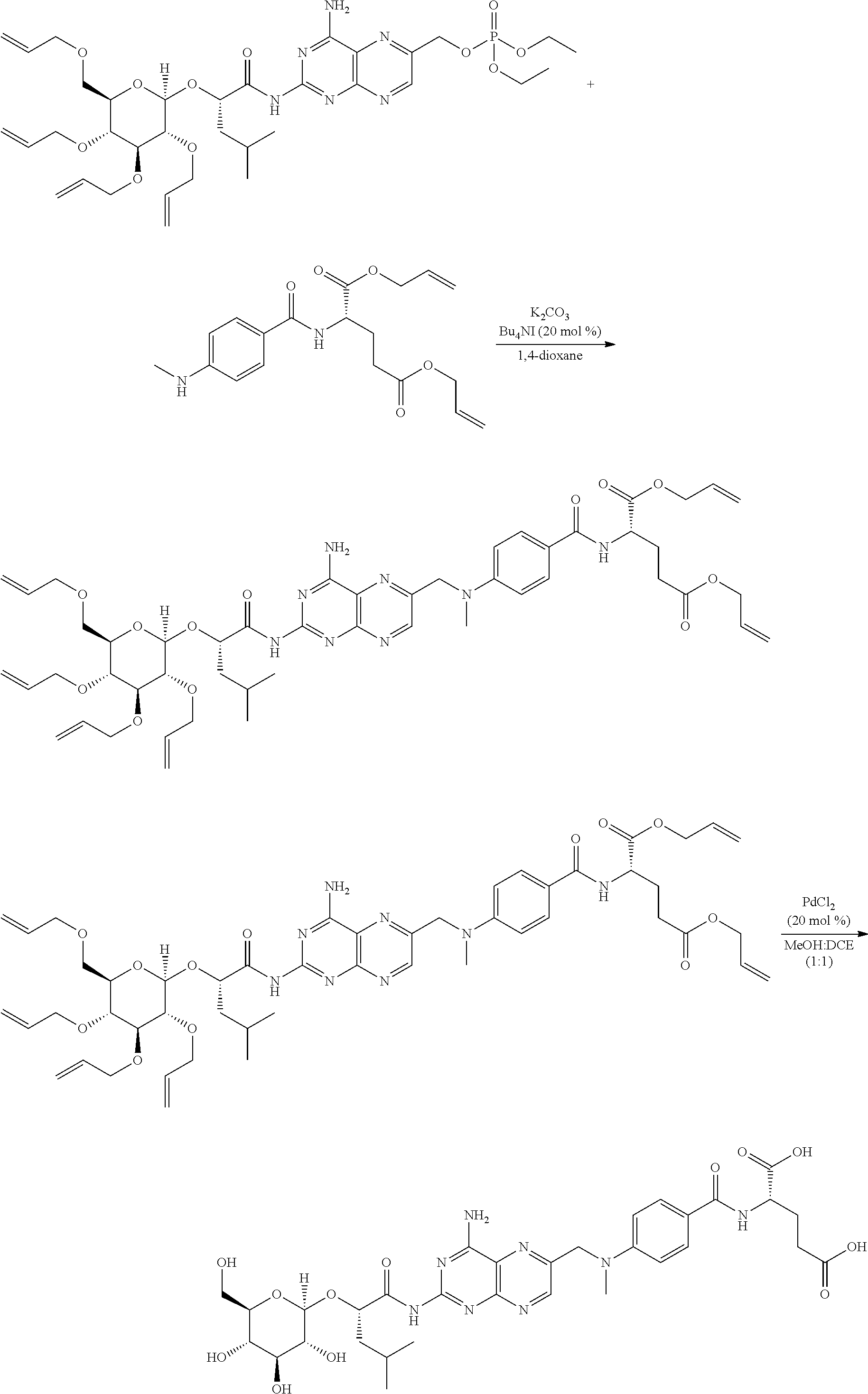

### Step 1

### (2R,3R,4S,5R,6R)-3,4,5-Tris(allyloxy)-6-[(allyloxy)methyl]-2-bromotetrahydro-2H-pyran

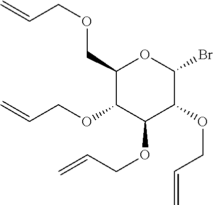 [SMILES: C=CCOC[C@H]1O[C@H](Br)[C@H](OCC=C)[C@@H](OCC=C)[C@@H]1OCC=C]

124.97 g (514.18 mmol, 1 equiv) of (2R,3R,4S,5S,6R)-2-Bromo-6-(hydroxymethyl)tetrahydro-2H-pyran-3,4,5-triol (provided via its previously reported synthesis from: Anqing Qichuang Pharmaceutical Co., Ltd.; Wu Xueping; Hai Wei CN108276396, 2018, A) is dissolved in 200 mL of methanol, and 745 g (6.16 mol, 12 equiv) allyl bromide and 1.71 Kg (6.16 mol, 12 equiv) of ferrous sulfate is added. The solution is heated to 60 C. After TLC determines the starting material has been exhausted, the reaction is cooled to room temperature, and the iron(II) sulphite precipitate is removed via filtration. The filtrate is concentrated in vacuo and the crude (2R,3R,4S,5R,6R)-3,4,5-Tris(allyloxy)-6-[(allyloxy)methyl]-2-bromotetrahydro-2H-pyran product is purified by column chromatography on silica gel.

### Step 2

### Cyanomethyl (S)-2-{(2S,3R,4S,5R,6R)-3,4,5-tris(allyloxy)-6-[(allyloxy)methyl]tetrahydro-2H-pyran-2-yloxy}-4-methylvalerate

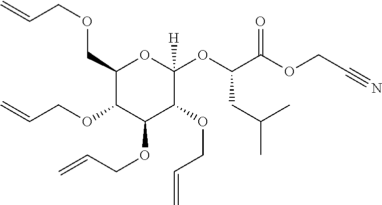 [SMILES: C=CCOC[C@H]1O[C@@H](O[C@@H](CC(C)C)C(=O)OCC#N)[C@H](OCC=C)[C@@H](OCC=C)[C@@H]1OCC=C]

To a solution of (2R,3R,4S,5R,6R)-3,4,5-Tris(allyloxy)-6-[(allyloxy)methyl]-2-bromotetrahydro-2H-pyran (121 g, 300 mmol, 1 equiv) in anhydrous DCM (200 mL) containing In(NTf₂)₃ (28.6 g, 30 mmol, 10 mol %) and 4 Å molecular sieves (15 g), Cyanomethyl (S)-2-hydroxy-4-methylvalerate (provided via its previously reported synthesis from: Tetrahedron Letters, 1999, vol. 40, #34, p. 6189-6192) (61 g, 360 mmol, 1.2 equiv) was added under Ar. The mixture was allowed to stir for 2 h at room temperature. The reaction mixture was evaporated in vacuo. The resulting residue was purified by rapid elution chromatography on silica gel to yield Cyanomethyl (S)-2-{(2S,3R,4S,5R,6R)-3,4,5-tris(allyloxy)-6-[(allyloxy)methyl]tetrahydro-2H-pyran-2-yloxy}-4-methylvalerate.

### Step 3

### (S)-2-{(2S,3R,4S,5R,6R)-3,4,5-Tris(allyloxy)-6-[(allyloxy)methyl]tetrahydro-2H-pyran-2-yloxy}-4-methylvaleric Acid

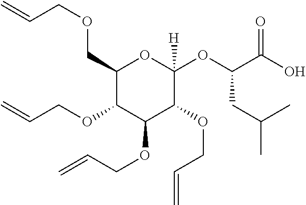 [SMILES: C=CCOC[C@H]1O[C@@H](O[C@@H](CC(C)C)C(=O)O)[C@H](OCC=C)[C@@H](OCC=C)[C@@H]1OCC=C]

A 1 L flask with a magnetic stirrer was charged with 160 mL of methanol, 80 mL of water and 1.75 g of (9.03 mol) of KOH. The solution was stirred at 23 C for 5 minutes. Cyanomethyl (S)-2-{(2S,3R,4S,5R,6R)-3,4,5-tris(allyloxy)-6-[(allyloxy)methyl]tetrahydro-2H-pyran-2-yloxy}-4-methylvalerate (8.6 g, 0.019 mol) was added to this solution at 23 C. The reaction was run until complete consumption of starting material was indicated by TLC. The solution was concentrated under reduced pressure. The pH of the solution was adjusted to 4.2 with dilute aqueous HCl and a solid precipitated. The solid was isolated by filtration and washed with water. Further purification was preformed via chromatography on silica gel.

### Step 4

### 2-(tert-Butoxycarbonylamino)-6-(hydroxymethyl)-3H-pteridin-4-one

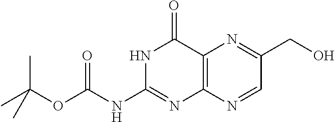 [SMILES: CC(C)(C)OC(=O)Nc1nc2ncc(CO)nc2c(=O)[nH]1]

To a mixture of 2-Amino-6-(hydroxymethyl)-3H-pteridin-4-one (50 g, 260 mmols, 1 equiv) (provided via its previously reported synthesis from: Chem. Ber. 113, 1514-1523 (1980)) and di-tert-butyl dicarbonate (68 g, 312 mmol, 1.2 equiv.) was added finely powdered La(NO₃)₃·6H2O (5 g, 13 mmol, 5 mol %) and the reaction mixture was stirred under solvent-free conditions at room temperature for 20 minutes. After completion of the reaction as monitored by TLC, water was added to the reaction mixture and the product was extracted into ethyl acetate three times. The combined organic layer was washed with brine, dried over anhydrous sodium sulfate, and concentrated under reduced pressure to give a crude product, which was purified by silica gel column chromatography to afford 2-(tert-Butoxycarbonylamino)-6-(hydroxymethyl)-3H-pteridin-4-one.

### Step 5

### 2-(tert-Butoxycarbonylamino)-6-(hydroxymethyl)-4-(1H-1,2,4-triazol-1-yl)pteridine

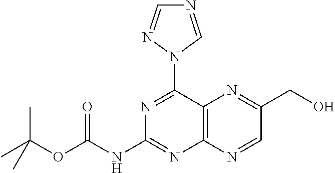 [SMILES: CC(C)(C)OC(=O)Nc1nc(-n2cncn2)c2nc(CO)cnc2n1]

2-(tert-Butoxycarbonylamino)-6-(hydroxymethyl)-3H-pteridin-4-one (40 g, 136 mmol, 1 equiv) and BOP (78.4 g 177 mmol, 1.3 equiv) were suspended in dry acetonitrile. DBU (31 g, 204 mmol, 1.5 equiv) was then added dropwise and the reaction mixture became homogeneous. After stirring for 10 min at room temperature, 1,2,4-triazole (14 g, 204 mmol 1.5 equiv) was added dropwise and the solution was stirred for a further 48 h. The excess solvent was removed under reduced pressure and the resulting residue was partitioned between ethyl acetate and water. The organic layer was dried over MgSO₄ and purified by flash chromatography to yield 2-(tert-Butoxycarbonylamino)-6-(hydroxymethyl)-4-(1H-1,2,4-triazol-1-yl)pteridine as a pale-yellow solid.

### Step 6

### 2-(tert-Butoxycarbonylamino)-6-(diethoxyphosphinooxymethyl)-4-(1H-1,2,4-triazol-1-yl)pteridine

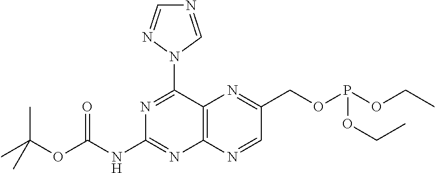 [SMILES: CCOP(OCC)OCc1cnc2nc(NC(=O)OC(C)(C)C)nc(-n3cncn3)c2n1]

To a flame-dried 250 mL RB flask under argon atmosphere was added 2-(tert-Butoxycarbonylamino)-6-(hydroxymethyl)-4-(1H-1,2,4-triazol-1-yl)pteridine (15 g, 44 mmol, 1 equiv) followed by addition of DCM (120 mL) and anhydrous NMI (5.2 mL 5 g, 61 mmol, 1.4 equiv). To this mixture was added diethyl phosphoryl chloride (7.6 mL, 9.1 g, 53 mmol, 1.2 equiv) in several portions with stirring. The reaction turned light yellow in color and was complete by TLC in 2 to 3 hours. The reaction was then concentrated in vacuo and purified by flash chromatography to afford 2-(tert-Butoxycarbonylamino)-6-(diethoxyphosphinooxymethyl)-4-(1H-1,2,4-triazol-1-yl)pteridine.

### Step 7

### 6-(Diethoxyphosphinooxymethyl)-4-(1H-1,2,4-triazol-1-yl)-2-pteridinylamine

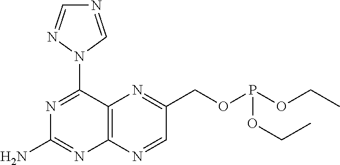 [SMILES: CCOP(OCC)OCc1cnc2nc(N)nc(-n3cncn3)c2n1]

2-(tert-Butoxycarbonylamino)-6-(diethoxyphosphinooxymethyl)-4-(1H-1,2,4-triazol-1-yl)pteridine (10 g) was dissolved in 4M hydrochloric acid in 1,4-dioxane (70 mL) and stirred at room temperature for 1 hr. The solvent was evaporated in vacuo and the residue diluted with saturated sodium bicarbonate (20 mL) and 1 M NaOH (10 mL), extracted with EtOAc (2×30 mL), dried over magnesium sulfate, filtered, and evaporated to dryness in vacuo to provide crude 6-(Diethoxyphosphinooxymethyl)-4-(1H-1,2,4-triazol-1-yl)-2-pteridinylamine, which was further purified by flash chromatography on silica gel.

### Step 8

### N-[6-(Diethoxyphosphinooxymethyl)-4-(1H-1,2,4-triazol-1-yl)-2-pteridinyl](S)-2-{(2S,3R,4S,5R,6R)-3,4,5-tris(allyloxy)-6-[(allyloxy)methyl]tetrahydro-2H-pyran-2-yloxy}-4-methylvaleramide

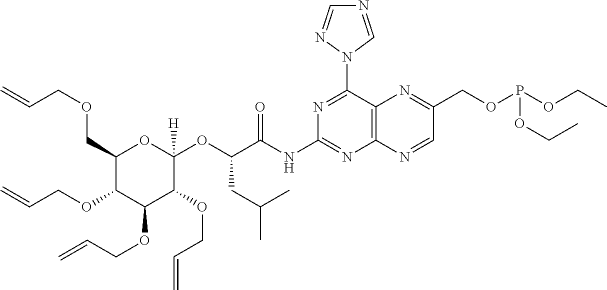 [SMILES: C=CCOC[C@H]1O[C@@H](O[C@@H](CC(C)C)C(=O)Nc2nc(-n3cncn3)c3nc(COP(OCC)OCC)cnc3n2)[C@H](OCC=C)[C@@H](OCC=C)[C@@H]1OCC=C]

To a solution of 6-(Diethoxyphosphinooxymethyl)-4-(1H-1,2,4-triazol-1-yl)-2-pteridinylamine 3 g, 11 mmol, 1 eq) in THF (12 mL) was added DCC (3.4 g, 16.5 mmol, 1.5 eq) and HOBt (2.2 g, 16.5 mmol, 1.5 eq). After stirring for 10 min, (S)-2-{(2S,3R,4S,5R,6R)-3,4,5-Tris(allyloxy)-6-[(allyloxy)methyl]tetrahydro-2H-pyran-2-yloxy}-4-methylvaleric acid (5.5 g, 12 mmol, 1.1 eq) was added. The mixture was stirred at 25° C. for 16 hours. The reaction mixture was concentrated in vacuo. The crude product was purified by reverse-phase HPLC and lyophilized to yield N-[6-(Diethoxyphosphinooxymethyl)-4-(1H-1,2,4-triazol-1-yl)-2-pteridinyl](S)-2-{(2S,3R,4S,5R,6R)-3,4,5-tris(allyloxy)-6-[(allyloxy)methyl]tetrahydro-2H-pyran-2-yloxy}-4-methylvaleramide.

### Step 9

### N-[4-Amino-6-(diethoxyphosphinooxymethyl)-2-pteridinyl](S)-2-{(2S,3R,4S,5R,6R)-3,4,5-tris(allyloxy)-6-[(allyloxy)methyl]tetrahydro-2H-pyran-2-yloxy}-4-methylvaleramide

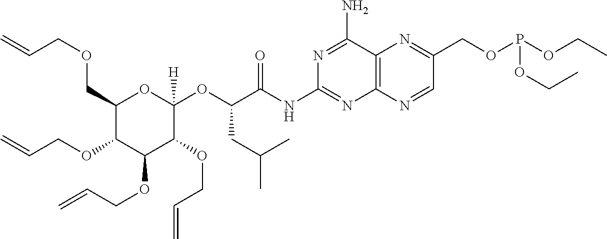 [SMILES: C=CCOC[C@H]1O[C@@H](O[C@@H](CC(C)C)C(=O)Nc2nc(N)c3nc(COP(OCC)OCC)cnc3n2)[C@H](OCC=C)[C@@H](OCC=C)[C@@H]1OCC=C]

Saturated aqueous ammonium hydroxide (3 mL) was added to a suspension of (4 g, 5 mmol, 1 equivalent) in dioxane (20 ml). The resulting thick suspension was stirred at room temperature until all the starting material was consumed as determined by TLC. The solution was concentrated under reduced pressure, and the pale-yellow solid which precipitated collected by filtration and washed with water before being dried in vacuo giving N-[4-Amino-6-(diethoxyphosphinooxymethyl)-2-pteridinyl](S)-2-{(2S,3R,4S,5R,6R)-3,4,5-tris(allyloxy)-6-[(allyloxy)methyl]tetrahydro-2H-pyran-2-yloxy}-4-methylvaleramide, which was used without further purification.

### Step 10

### N-{4-Amino-6-[(diethoxyphosphoryloxy)methyl]-2-pteridinyl}(S)-2-{(2S,3R,4S,5R,6R)-3,4,5-tris(allyloxy)-6-[(allyloxy)methyl]tetrahydro-2H-pyran-2-yloxy}-4-methylvaleramide

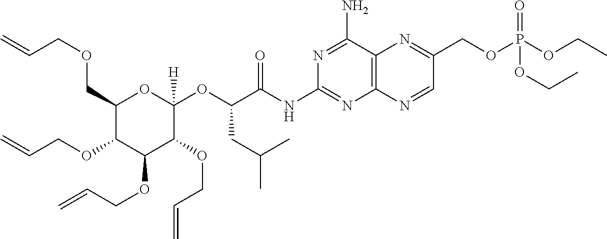 [SMILES: C=CCOC[C@H]1O[C@@H](O[C@@H](CC(C)C)C(=O)Nc2nc(N)c3nc(COP(=O)(OCC)OCC)cnc3n2)[C@H](OCC=C)[C@@H](OCC=C)[C@@H]1OCC=C]

N-[4-Amino-6-(diethoxyphosphinooxymethyl)-2-pteridinyl](S)-2-{(2S,3R,4S,5R,6R)-3,4,5-tris(allyloxy)-6-[(allyloxy)methyl]tetrahydro-2H-pyran-2-yloxy}-4-methylvaleramide (2.0 g, 2.7 mmol, 1 equiv) was dissolved in dichloromethane (10 mL) and tert-butyl hydroperoxide [TBHP of decane solution (5.0-6.0 M, 540 μL, 1 equiv)] was added at 23C and stirred for 70 minutes. The solvent of the resulting solution was evaporated in vacuo, and the residue was purified by column chromatography to give N-{4-Amino-6-[(diethoxyphosphoryloxy)methyl]-2-pteridinyl}(S)-2-{(2S,3R,4S,5R,6R)-3,4,5-tris(allyloxy)-6-[(allyloxy)methyl]tetrahydro-2H-pyran-2-yloxy}-4-methylvaleramide.

### Step 11

### Diallyl (S)-2-[p-(methylamino)benzylamino]glutarate

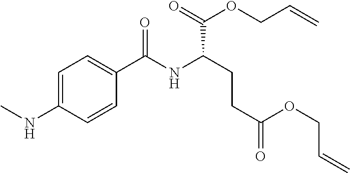 [SMILES: C=CCOC(=O)CC[C@H](NC(=O)c1ccc(NC)cc1)C(=O)OCC=C]

A 1 L RB flask was equipped with a magnetic stirrer, thermometer and condenser. The flask was charged with 42 mL of DMF, 5.0 g (15 mmol, 1 equiv) of N-[4-(methylamino)benzoyl]-L-glutamic acid disodium salt (provided via its previously reported synthesis from: Patent WO2012/074496 A1 PCT/TR2010/00023) and 4.3 mL of (6.0 g, 49 mmol, 3.2 equiv) of allyl bromide at 23C. The suspension was stirred at 60C for 5 hours. The solution was cooled to room temperature and 42 mL of water was added. The mixture was stirred for 20 minutes, and a white precipitate formed. The solid was isolated via filtration and washed with 20 mL of water. The solid was dried under vacuum at 50C for 3 hours yielding Diallyl (S)-2-[p-(methylamino)benzylamino]glutarate as a white solid, which was used without further purification.

### Step 12

### Diallyl (S)-2-{p-[({2-[(S)-2-{(2S,3R,4S,5R,6R)-3,4,5-tris(allyloxy)-6-[(allyloxy)methyl]tetrahydro-2H-pyran-2-yloxy}-4-methylvalerylamino]-4-amino-6-pteridinyl}methyl)-N-methylamino]benzylamino}glutarate

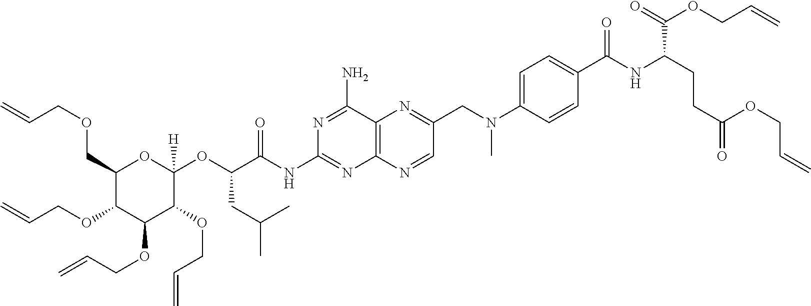 [SMILES: C=CCOC[C@H]1O[C@@H](O[C@@H](CC(C)C)C(=O)Nc2nc(N)c3nc(CN(C)c4ccc(C(=O)N[C@@H](CCC(=O)OCC=C)C(=O)OCC=C)cc4)cnc3n2)[C@H](OCC=C)[C@@H](OCC=C)[C@@H]1OCC=C]

To a 25 mL R^{B} flask was added N-{4-Amino-6-[(diethoxyphosphoryloxy)methyl]-2-pteridinyl}(S)-2-{(2S,3R,4S,5R,6R)-3,4,5-tris(allyloxy)-6-[(allyloxy)methyl]tetrahydro-2H-pyran-2-yloxy}-4-methylvaleramide (1 g, 1.3 mmol, 1 equiv) followed by addition of tetrabutylammonium iodide (97 mg, 0.26 mmol, 0.2 equiv), K₂CO₃ (539 mg, 3.9 mmol, 3 equiv), and 1,4-dioxane (5.0 mL 0.26 M). The suspension was stirred rapidly for 5 minutes, then diallyl (S)-2-[p-(methylamino)benzylamino]glutarate (515 mg, 1.4 mmol, 1.1 equiv) was added as a single portion. The reaction was heated to 80° C. with stirring. The reaction was followed by TLC until all of the starting material was consumed, then the reaction was cooled to 23C diluted with 20 mL of diethyl ether to encourage precipitating inorganic materials, and then filtered through a small plug of silica gel with copious rinse of a 1:1 mixture of hexanes and ethyl acetate. The filtrate was concentrated in vacuo and purified by chromatography on silica gel to give Diallyl (S)-2-{p-[({2-[(S)-2-{(2S,3R,4S,5R,6R)-3,4,5-tris(allyloxy)-6-[(allyloxy)methyl]tetrahydro-2H-pyran-2-yloxy}-4-methylvalerylamino]-4-amino-6-pteridinyl}methyl)-N-methylamino]benzylamino}glutarate.

### Step 13

### (S)-2-(p-{[(2-{(S)-2-[(2S,3R,4S,5S,6R)-3,4,5-Trihydroxy-6-(hydroxymethyl)tetrahydro-2H-pyran-2-yloxy]-4-methylvalerylamino}-4-amino-6-pteridinyl)methyl]-N-methylamino}benzylamino)glutaric Acid

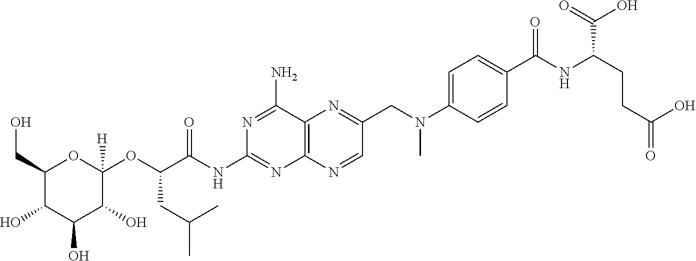 [SMILES: CC(C)C[C@H](O[C@@H]1O[C@H](CO)[C@@H](O)[C@H](O)[C@H]1O)C(=O)Nc1nc(N)c2nc(CN(C)c3ccc(C(=O)N[C@@H](CCC(=O)O)C(=O)O)cc3)cnc2n1]

PdCl₂ (22 mg, 0.10 mmol, 20 mol %) was added to a solution of Diallyl (S)-2-{p-[({2-[(S)-2-{(2S,3R,4S,5R,6R)-3,4,5-tris(allyloxy)-6-[(allyloxy)methyl]tetrahydro-2H-pyran-2-yloxy}-4-methylvalerylamino]-4-amino-6-pteridinyl}methyl)-N-methylamino]benzylamino}glutarate (500 mg, 515 umols, 1 equiv) in MeOH (5 mL) and 1,2-DCE (5 mL) and the combined mixture was heated to 70° C. The reaction was followed by TLC and once all the starting material was exhausted, the solvent was evaporated in vacuo and the residue subjected to chromatography on silica gel to yield (S)-2-(p-{1[(2-{(S)-2-[(2S,3R,4S,5S,6R)-3,4,5-Trihydroxy-6-(hydroxymethyl)tetrahydro-2H-pyran-2-yloxy]-4-methylvalerylamino}-4-amino-6-pteridinyl)methyl]-N-methylamino}benzylamino)glutaric acid as a light yellow solid.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. An aberrant immune cell-selective compound, or a pharmaceutically acceptable salt thereof, comprising:
a Bait comprising a ligand moiety that binds an enzyme or receptor overexpressed by the aberrant immune cell relative to a normal immune cell, or is not expressed or is expressed at lower levels by a normal immune cell, or having more activity than the enzyme or receptor has in a normal immune cell, the aberrant immune cell being associated with an immune-related disorder; and
a Payload comprising a therapeutic moiety that reduces an activity of, inhibits the proliferation of, or kills, the aberrant immune cell.

2. The compound of claim 1, wherein the Bait comprises a ligand that binds the receptor on the immune cell.

3. The compound of claim 1, wherein the Bait comprises a ligand that binds the enzyme of the immune cell.

4. The method of claim 3, wherein the ligand comprises a substrate of the enzyme.

5. The compound of claim 4, wherein the substrate comprises a peptide bond.

6. The compound of claim 3, wherein enzyme is a matrix metalloprotease (MMP).

7. The compound of claim 1, wherein compound has the structure
Bait----Payload,
wherein the Bait comprises a Cap attached to a Subunit.

8. The compound of claim 7, wherein the Cap is selected to the group consisting of 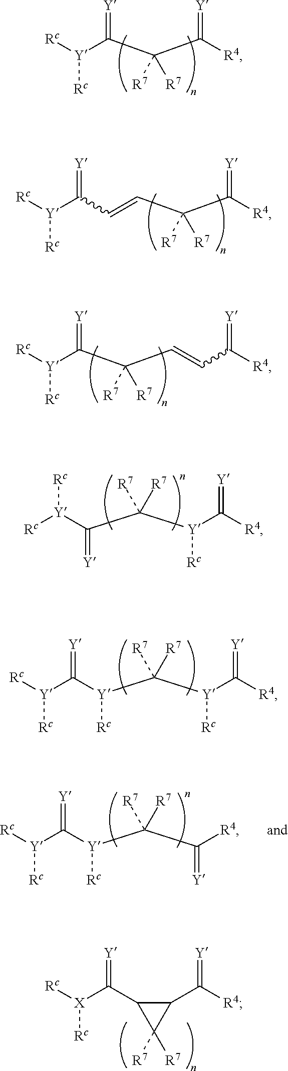
wherein R⁴ is independently, at each occurrence, a bond to any R⁸ or a bond to an R⁶ in a Subunit.

9. The compound of claim 7, wherein the Subunit is selected to the group consisting of 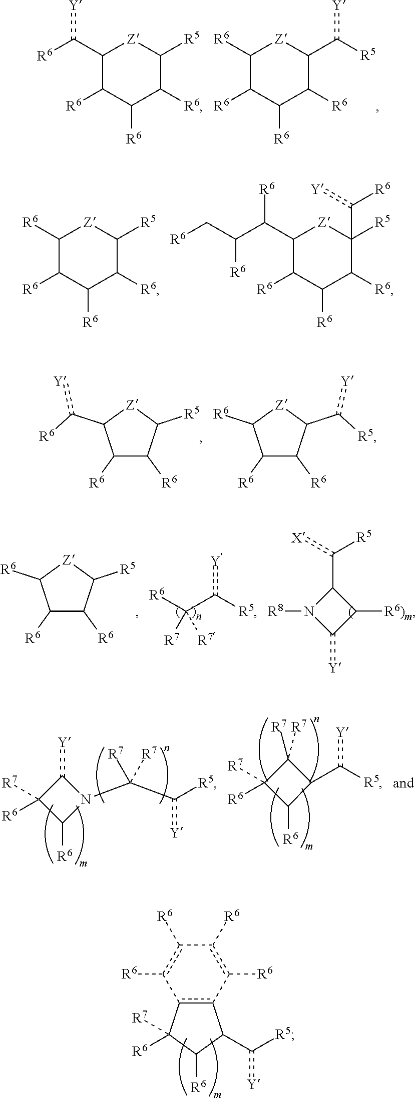
wherein:
--- is an optional single bond;
=== is an optional single or double bond;
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to an R⁶ in another Subunit, a bond to an R⁸ in another Subunit, or a bond to the Payload;
R⁶ is independently, at each occurrence, selected from the group consisting of H, —R^{D}, —R^{A}—R⁵ wherein R⁵ is in another Subunit, —R^{A}—R⁴, where R⁴ is in a Cap, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with a one, two, three, or four halo(s);
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁₀ aryl, a 5-10-membered heteroaryl, C₁₋₄ alkyl, —(CH₂)₁₋₃—C₆₋₁₀ aryl, and —(CH₂)₁₋₃-5-10 membered heteroaryl,
wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵ in another Subunit, or a bond to R⁴ in a Cap;
m is 0, 1, 2, 3, or 4; and
n is 0, 1, 2, or 3.

10. The compound of claim 4, wherein the Bait comprises a substrate of an MMP, the substrate comprising N-succinyl-alanine-alanine-phenylalanine (SAAF), N-glutaryl-alanine-alanine-phenylalanine (GAAF)-, N-succinyl-alanine-alanine-leucine (SAAL)-, or N-glutaryl-alanine-alanine-leucine (GAAL), N-malonyl-alanine-alanine-phenylalanine (MAAF), or N-malonyl-alanine-alanine-leucine (MAAL), alanine-alanine-phenylalanine (AAP), alanine (A), methionine (M), 7-methoxy, glycine-R, or L-leucine.

11. The compound of claim 1, wherein the Bait comprises an immunoglobulin or binding fragment thereof.

12. The compound of claim 11, wherein the immunoglobulin comprises an antibody or specific binding fragment thereof, a camelid, a single domain immunoglobulin, a chimeric antigen receptor (CAR), or a CAR T cell.

13. The compound of claim 12, wherein the antibody comprises a human antibody, a recombinant antibody, a humanized antibody, a bispecific antibody, or a monoclonal antibody.

14. The compound of claim 1, wherein the Bait comprises a ligand conjugated to a second ligand.

15. The compound of claim 14, wherein the second ligand is specific for a protein different than the enzyme or receptor to which the first ligand binds.

16. The compound of claim 1, wherein the Bait comprises a proBait.

17. The compound of claim 1, wherein the Payload comprises a therapeutic moiety comprising a small molecule.

18. The compound of claim 17, wherein the small molecule comprises a metabolic inhibitor, a small molecule cytotoxic agent, or a small molecule inhibitor of an immune cell receptor.

19. The compound of claim 18, wherein the therapeutic moiety comprises a prodrug.

20. The compound of claim 1, wherein the Payload comprises a therapeutic moiety comprising a polynucleotide that inhibits or reduces the expression of an immune cell metabolic enzyme or receptor.

21. The compound of claim 20, wherein the polynucleotide comprises an antisense polynucleotide, an miRNA, or an siRNA.

22. The compound of claim 1, wherein the Payload comprises a therapeutic moiety comprising an immunoglobulin, or binding fragment thereof.

23. The compound of claim 22, wherein the therapeutic moiety comprises an immunoglobulin comprising an antibody, a camelid, a single domain immunoglobulin, or a CAR.

24. The compound of claim 23 wherein the therapeutic moiety comprises an antibody that is a human antibody, a recombinant antibody, a humanized antibody, a monoclonal antibody, a bispecific antibody, a monoclonal antibody, an antibody-drug conjugate, or a binding fragment thereof.

25. The method of claim 24, wherein the antibody comprises an anti-TNF antibody, an anti-CD19 antibody, anti-CD22 antibody, an anti-CD80 antibody, an anti-CD86 antibody, an anti-IL-6 receptor antibody, an anti-Interleukin-1 type I receptor (IL-1RI) antibody, an anti-IL-12/23 antibody, an anti-CD20 antibody, or an anti-IL-17 antibody,

26. The compound of claim 24, wherein the immunoglobulin comprises an antibody-drug conjugate

27. The compound of claim 26, wherein the immunoglobulin or binding fragment thereof is conjugated to a second therapeutic moiety.

28. The compound of claim, 27, wherein the second therapeutic moiety comprises a small molecule cytotoxic agent, a small molecule inhibitor of a metabolic enzyme or receptor, or a small molecule inhibitor of an immune cell receptor.

29. The compound of claim 27, wherein the immunoglobulin is conjugated to a second immunoglobulin or binding fragment thereof.

30. The compound of claim 29, wherein the second immunoglobulin comprises a human antibody, a recombinant antibody, a humanized antibody, a bispecific antibody, a monoclonal antibody, a camelid, or a single domain immunoglobulin.

31. The compound of claim 30, wherein the second immunoglobulin comprises an anti-TNF antibody, an anti-CD19 antibody, anti-CD22 antibody, an anti-CD80 antibody, an anti-CD86 antibody, an anti-IL-6 receptor antibody, an anti-Interleukin-1 type I receptor (IL-1RI) antibody, an anti-IL-12/23 antibody, an anti-CD20 antibody, or an anti-IL-17 antibody.

32. A formulation comprising the compound of any one of claims 1-31 and a pharmaceutically acceptable carrier.

33. A method of treating, or decreasing a risk of protracting, an immune-related disorder in a subject, comprising administering to the subject an amount of the compound of any one of claims 1-31 or the formulation of claim 32 effective to reduce a symptom of, or to reduce the risk of protracting, the immune-related disorder.

34. The method of claim 33 wherein the immune-related disorder is an inflammatory disorder.

35. The method of claim 33, wherein the disorder is systemic lupus erythematosus (SLE), rheumatoid arthritis (RA), multiple sclerosis (MS), pemphigus vulgaris, bullous pemphigoid, scleroderma, myasthenia gravis, psoriasis, autoimmune myositis, or infection by SARS-CoV2.

36. Use of the compound of claims 1-31 to reduce a symptom of an immune-related disorder in a subject, or to reduce the risk of a subject contracting an immune-related disorder
